(19) United States
(12) Patent Application Publication
(10) Pub. No.: US 2005/0009124 A1
(43) Pub. Date: Jan. 13, 2005

(54) Assays for detection of phosphoinositide kinase and phosphatase activity

(75) Inventors: Beth E. Drees, Park City, UT (US); Paul O. Neilsen, Draper, UT (US); Angie M. Branch, West Jordan, UT (US); Amber Weipert, Draper, UT (US); Heather A. Hudson, Park City, UT (US); Li Feng, Salt Lake City, UT (US); Glenn Prestwich, Salt Lake City, UT (US)

Correspondence Address: THORPE NORTH & WESTERN, LLP., 8180 SOUTH 700 EAST, SUITE 200, P.O. BOX 1219, SANDY, UT, 84070, US

(73) Assignee: Echelon Biosciences Incorporated

(21) Appl. No.: 10/850,833

(22) Filed: May 20, 2004

Related U.S. Application Data
(63) Continuation-in-part of application No. 09/991,933, filed on Nov. 26, 2001.
(63) Continuation-in-part of application No. 10/712,073, filed on Nov. 13, 2003.
(60) Provisional application No. 60/426,572, filed on Nov. 15, 2002.

(51) Int. Cl. G01N 33/53

(52) U.S. Cl. 435/7.92

(57) ABSTRACT

A lipid assay kit for detection of a lipid kinase or phosphatase and method of use thereof. The assay is preferably a competitive assay wherein the product lipid has a stronger affinity to the lipid detector protein than the substrate lipid. The lipid recognition motif is preferably a pleckstrin homology (PH) domain.

This application is a continuation-in-part based on co-pending U.S. patent application Ser. No. 09/991,933 filed on Nov. 26, 2001 and U.S. patent application Ser. No. 10/712,073 filed on Nov. 13, 2003 which claims the benefit of U.S. Provisional Application No. 60/426,572, filed on Nov. 15, 2002.

### BACKGROUND OF THE INVENTION

1. Field of the Invention

The present invention relates to enzymes that phosphorylate inositol lipids or dephosphorylate inositol lipids. More particularly, the present invention relates to detection methods, kits, and apparatuses for the detection of such enzymes.

Phosphoinositide phosphates are important second messengers in signaling pathways governing cellular proliferation, survival, morphology, and motility. There is described herein the development of assay methods for determination of phosphoinositide phosphatase activity. Although phosphatidylinositol phosphates (phosphoinositides) are minor components of cellular membranes, phosphoinositide-dependent signaling pathways play central roles in the regulation of many cellular processes.(Martin, Ann. Rev. Cell Dev. Biol., 14:231-2614 (1998)) Phosphoinositides are distinctive among phospholipids in their ability to be quickly modified by phosphorylation or dephosphorylation of their inositol headgroups, and thus to act as second messengers which regulate site-specific signaling and assembly of membrane-associated protein complexes. Because the activity of PIP second messengers is determined by their phosphorylation state, the enzymes that act to modify these lipids are central to the correct execution of these signaling events.(Leslie, Chemical Reviews, 101:2365-2380 (2001)) Phosphoinositide biosynthesis occurs through the interplay of lipid-specific kinases and phosphatases, as shown in FIG. 1. These enzymes act to add or remove phosphate groups from the 3′, 4′, or 5′ position of the inositol ring, examples shown in FIG. 1 are PI 3-Kinase (PI 3-K) and PTEN phosphatase. The phosphorylation state of phosphoinositides is the main determinate of their biological activity. In addition, phosphoinositides may also be hydrolysed to form inositol phosphates and diacylglycerol through the activity of phospholipases, as is also shown.

Phosphoinositides are key lipid second messengers in cellular signaling, with phosphatidylinositol (PI) dependent signaling pathways playing central roles in the regulation of many cellular processes. Disruption of these pathways is common to many disease states, including inflammation, diabetes, cardiovascular disease, and cancer. Because the activity of PI second messengers is determined by their phosphorylation state, the enzymes that act to modify these lipids are central to the correct execution of PI dependent signaling pathways.

Of all lipid messengers, phosphoinositides are the most diverse. They are generated by phosphorylation and dephosphorylation at the D3, D4, and D5 hydroxyls of the inositol head group by specific kinases and phosphatases. Activation of cellular signaling pathways often results from specific phosphoinositide production in response to a stimulus. The concentration and location of phosphoinositide are tightly regulated by a multitude of kinases, phosphatases, and hence produce lipid signals for temporal and spatial regulation of diverse cellular processes. Different phosphoinositides have specific roles in signaling pathways, cytoskeletal architecture, or membrane trafficking for a given cell-type. Table B-1 shows selected functions of the seven phosphoinositides and phosphatidylinositol (PI).

TABLE B-1
Biological roles of phosphoinositides in cell signaling

| PIPₙ | Biological Role |
|---|---|
| PI | Substrate for PI 3-K, PITP, PI 4-Ks and PI 5-K |
| PI(3)P | Interacts with FYVE domains and PX domains and is a substrate for 5- |
| | kinase. Role in vesicular protein sorting, vacuole function, membrane |
| | trafficking, endosomal sorting and cytoskeletal regulation |
| PI(4)P | Substrate for 5- and 3-kinase, role in protein secretion; affinity for dynamin |
| | and inhibition of a PI(3, 4, 5)P₃ 5-phosphatase; vesicle coat formation |
| | (COPII, COPI); maintenance of Golgi structure and formation of secretory |
| | vesicles |
| PI(5)P | Substrate for 3- and 4-kinases |
| PI(3, 4)P₂ | Product of 5-phosphatase action on PI(3, 4, 5)P₃. Stimulation of PKB, PKC |
| | kinases and platelet protein kinases, and promotion of cell survival; binds to |
| | PX domain of p47^{phox} |
| PI(3, 5)P₂ | Vesicular protein sorting, normal vacuolar function and stress response |
| PI(4, 5)P₂ | Substrate for PLC, PI 3-K, activator of ARF, PLD; recruitment of |
| | membrane proteins via PH domain; binding to profilin, gelsolin, tau; |
| | endocytosis via AP180 |
| PI(3, 4, 5)P₃ | Recruits PH-domains to signaling complexes; targets include: PKB/Akt, |
| | PKC, PDK1, Btk, GRP-1. Activator of ARF GAP activity via centaurin and |
| | GCS-1 |

In particular, phosphatidylinositol 3-kinase (PI 3-Kinase) is important in pathways mediating cell proliferation, survival, differentiation and motility. Inhibitors of PI 3-Kinase have been used to confirm the cellular functions of PI 3-Kinase; but thus far, such inhibitors have not been deemed suitable for therapeutic use because of problems such as toxicity and low selectivity. The PI 3-Kinase of heterodimeric lipid kinases is known primarily for its involvement in the phosphorylation of inositol lipids via transfer of the .gamma. phosphate of ATP to the D-3 position of the inositol ring of PI, PI(4)P, and PI(4,5)P₂ giving rise to PI(3)P, PI(3,4)P₂, and PI(3,4,5)P₃ respectively. FIG. 1 shows an overview of these phosphoinositide metabolic pathways. PI(4,5)P₂ is a minor component of the plasma membrane's inner leaflet, and is part of a second messenger system that transduces many hormone signals. When not effected by PI 3-Kinase, the PI(4,5)P₂ pathway includes a receptor with seven transmembrane segments, a heterotrimeric G-protein, and a specific protein kinase phospholipase C (PLC). Ligand binding to the receptor activates the G protein, G.sub.q, whose membrane-anchored alpha subunit in complex with GTP diffuses laterally along the plasma membrane to activate the membrane-bound PLC. As shown in FIG. 1, the activated PLC catalyzes the hydrolysis of PI(4,5)P₂ at its glycero-phospho bond, yielding inositol-1,4,5-trisphosphate (Ins(1,4,5)P₃ and diacylglycerol (DG).

PI 3-Kinase can be activated by tyrosine kinase receptors in response to growth factor stimulation. As discussed above, PI 3-Kinase is then involved in catalyzing the formation of PI(3,4,5)P₃ via phosphorylation of its substrate PI(4,5)P₂. By increasing cellular levels of PI(3,4,5)P₃, PI 3-Kinase induces the formation of defined molecular complexes that act in signal transduction pathways. Notably, PI 3-Kinase activity suppresses apoptosis and promotes cell survival through activation of its downstream target, PKB/Akt. PI(3,4,5)P₃ signaling is regulated by its formation and by its conversion into PI(4,5)P₂. The lipid phosphatases PTEN and SHIP are two enzymes that both act to decrease the cellular levels of PI(3,4,5)P₃ by conversion either to PI(4,5)P₂ or PI(3,4)P₂.

The myotubularin family of 3′ lipid phosphatases act on PI(3)P and/or PI(3,5)P₂ as their substrates and play a role in vesicle trafficking and autophagy (Taylor, et al., Proc Natl Acad Sci USA, 97:8910-5. (2000)). PI(3)P is generated by the activity of type III PI 3-Kinases, which have specific activity toward PI and have physiological roles distinct from type I PI 3-Kinases, which act in growth regulation and signaling. PI(3)P is important for vesicle trafficking, and the myotubularins may be important in renewing the pool of PI needed for these processes to be in balance.

The 5′ lipid phosphatase SHIP1 (SH2-containing inositol phosphatase 1) acts as a negative regulator of cytokine signaling and immune cell activation and differentiation (Bolland, et al., Immunity, 8:509-16. (1998), Liu, et al., J Exp Med, 188:1333-42. (1998), Brauweiler, et al., J Exp Med, 191:1545-54. (2000), Brauweiler, et al., Immunol Rev, 176:69-74. (2000), Rohrschneider, et al., Genes Dev, 14:505-20. (2000)), and regulates differentiation and maintenance of hematopoetic cell lineages.(Liu, et al., Blood, 91:2753-9. (1998)). SHIP1 is a 5′ lipid phosphatase which converts PI(3,4,5)P₃ to PI(3,4)P₂.(Damen, et al., Proc Natl Acad Sci USA, 93:1689-93. (1996, Lioubin, et al., Genes Dev, 10:1084-95. (1996))SHIP1 is a 5′ lipid phosphatase which converts PI(3,4,5)P₃ to PI(3,4)P₂.(Damen, et al., Proc Natl Acad Sci USA, 93:1689-93. (1996, Lioubin, et al., Genes Dev, 10: 1084-95. (1996)) SHIP1-regulated signaling is important for modulation of allergic responses. SHIP1 hydrolysis of PI(3,4,5)P₃ modulates PI 3-Kinase signaling to set the threshold for mast cell degranulation through its interaction with the immune inhibitory receptor FcγRIIB (Huber, et al., Proc Natl Acad Sci USA, 95:11330-5. (1998)). Thus, modulation of SHIP1 activity may be an avenue for therapeutic intervention in the treatment of allergic and other immune disorders.

The regulation of PI(3,4,5)P₃ levels is often defective in tumorigenesis.(Carpenter and Cantley, Biochim Biophys Acta, 1288:M11-6. (1996), Roymans and Slegers, Eur J Biochem, 268:487-98. (2001)) Elevated PI(3,4,5)P₃ levels contribute to cancer progression through constitutive activation of PKB/Akt (Franke, et al., Cell, 81:727-36. (1995), King, et al., Mol Cell Biol, 17:4406-18. (1997)), which provides a cell survival signal that blocks apoptosis and promotes survival following growth factor withdrawal or detachment from the extracellular matrix.(Franke, et al., Cell, 88:435-7. (1997), Kim, et al., Faseb J, 15:1953-62. (2001)) Elevated levels of PI(3,4,5)P₃ can occur through amplification of PI 3-Kinase gene expression, as is seen in some cancers (Shayesteh, et al., Nat Genet, 21:99-102. (1999), Ma, et al., Oncogene, 19:2739-44. (2000)), or through alterations in the activity of the lipid phosphatases which are responsible for modulating PI(3,4,5)P₃ levels.

PTEN (Phosphatase and Tensin Homolog deleted on Chromosome 10), also designated MMAC1 (Mutated in Multiple Advanced Cancers), is a 3′ phosphoinositide phosphatase that converts PI(3,4,5)P₃ to PI(4,5)P₂. (Maehama and Dixon, J Biol Chem, 273:13375-8. (1998), Maehama and Dixon, Trends Cell Biol, 9:125-8. (1999)) By converting PI(3,4,5)P₃ to PI(4,5)P₂, PTEN acts as a negative regulator of PKB/Akt activation by PI 3-Kinase.(Cantley and Neel, Proc Natl Acad Sci USA, 96:4240-5. (1999), Tamura, et al., J Biol Chem, 274:20693-703. (1999))

PTEN was identified as a tumor suppressor that is deleted or mutated in many cancer types.(Li, et al., Science, 275:1943-7. (1997), Teng, et al., Cancer Res, 57:5221-5. (1997)) Assays that accurately determine PTEN activity are very useful for both research and diagnostics purposes. In addition, if PI 3-Kinase targeted drug therapies are successfully developed for cancer treatment, determination of PTEN status could be a useful predictor of a tumor's response to treatment. In addition, alterations in SHIP1 activity may be associated with cancers of the blood. Recently, an inactivating mutation in the phosphatase catalytic domain of SHIP1 has been reported in primary myeloid leukemia cells(Luo, et al., Leukemia, 17:1-8. (2003)). SHIP1 is a negative regulator of myeloid cell survival, and loss of SHIP1 activity promotes cell survival and resistance to apoptosis, presumably through deregulation of PI 3-Kinase/Akt signaling (Liu, et al., Genes Dev, 13:786-91. (1999)). Thus, loss of SHIP1 activity may be a factor in the development of acute leukemia and chemotherapy resistance.

PI 3-Kinase activity and cellular levels of PI(3,4,5)P₃ are central to the control of cellular response to insulin stimulation and maintenance of glucose homeostasis.(Okada, et al., J Biol Chem, 269:3568-73. (1994), Czech and Corvera, J Biol Chem, 274:1865-8. (1999)) PI 3-Kinase-dependent signaling is particularly important for regulating several of the metabolic effects of insulin in muscle, such as translocation of the GLUT4 transporter to the plasma membrane. Defects in PI 3-Kinase response to insulin stimulation are associated with non-insulin dependent diabetes mellitus (NIDDM), or “type II” diabetes. Thus, modulation of PI(3,4,5)P₃ levels by phosphoinositide phosphatase activity is important in the signaling pathways governing insulin-regulated glucose metabolism, and could provide a possible point of intervention for treatment of NIDDM.

SHIP2 (SH2-containing inositol phosphatase 2) has recently emerged as a potential therapeutic target for modulating glucose metabolism in NIDDM and insulin resistance. SHIP2 is a second 5′ lipid phosphatase, closely related to SHIP 1, that also hydrolyzes PI(3,4,5)P₃ to produce PI(3,4)P₂. (Pesesse, et al., Biochem Biophys Res Commun, 239:697-700. (1997), Ishihara, et al., Biochem Biophys Res Commun, 260:265-72. (1999)) In contrast to SHIP1, SHIP2 is widely expressed in a variety of fibroblast and nonhematopoetic tumor cell lines,(Muraille, et al., Biochem J, 342 Pt 3:697-705. (1999)) and in particular is expressed in target tissues regulating insulin homeostasis.(Ishihara, et al., Biochem Biophys Res Commun, 260:265-72. (1999)) Tyrosine phosphorylation of SHIP2 occurs in response to treatment by a number of growth factors, including insulin, and is thought to act in the regulation of PI 3-Kinase signaling through insulin.(Habib, et al., J Biol Chem, 273:18605-9. (1998))

Another 5′ phosphoinositide phosphatase that appears to act in regulation of insulin response is SKIP (Skeletal muscle and kidney enriched inositol phosphatase), which is also highly expressed in insulin responsive tissues(Ijuin, et al., J. Biol. Chem., 275:10870-0875 (2000), Ijuin and Takenawa, Mol. Cell Biol., 23:1209-1220. (2003)). Similar to SHIP2, SKIP appears to play a role in negative regulation of PI 3-Kinase dependent responses to insulin stimulation, in particular glucose transport. SKIP inhibits PI 3-Kinase signaling by conversion of PI(3,4,5)P₃ to PI(3,4)P₂ and is a negative regulator of insulin-induced Akt activation, GLUT4 translocation, and cytoskeletal rearrangement. GLUT4 translocation is substantially inhibited by SKIP overexpression, and this effect is dependent on its 5′ phosphatase activity (Ijuin and Takenawa, Mol. Cell Biol., 23:1209-1220. (2003)). Thus, modulation of SKIP phosphatase activity may prove to be one approach to modulating insulin response in NIDDM.

In summary, the signaling pathways involving these lipid modifying enzymes are often perturbed in the events leading to disease, particularly in non-insulin dependent diabetes mellitus (NIDDM) and cancer. Based on the disclosure contained herein it is evident that the tools developed in the present invention have significant value for research and in diagnostic applications as well as for drug-screening platforms for identification of new lead molecules for therapeutic development.

Despite their importance, the potential of phosphoinositide modifying enzymes as targets for therapeutic development has not yet been fully realized. The identification of selective, drug-like inhibitors has been limited by the dearth of assay methods suitable for high throughput screening. The most common methods for assaying PI 3-Kinase activity involve phosphorylation of a PI or PI(4,5)P₂ substrate with ³²P-ATP, often requiring the formation of liposomes, followed by organic lipid extraction and separation and quantitation by radio-thin layer chromatography.

The most commonly used technique for determining phosphoinositide kinase or phosphatase activity is the malachite green assay for the determination of free phosphate generated by enzyme activity. While it is relatively easy to perform, this method has poor sensitivity (200 pmoles phosphate detection limit) and it is prone to interference from organic phosphates that might be present in buffers, cell-culture products, or biological samples. Other approaches for detection of phosphoinositide phosphate activity require enzymatic alteration of radiolabeled or fluorescently-labeled substrates. Detection of phosphatase activity is accomplished by thin layer chromatography to separate substrate and product. This approach is not readily accessible to many researchers, and is clearly not adaptable for high throughput screening(HTS) applications. Thus, there is a need for new reagents and assay formats for detection of phosphoinositide phosphatase activity. Ideally, these assays are non-radioactive, require minimal separation steps, and are specific for the phosphoinositide products of a particular enzymatic reaction. In addition, some assay formats should be homogenous and easily automated for HTS purposes.

A major advance in the understanding of phosphoinositide signaling has been the identification of a number of highly conserved modular protein domains that bind to various phosphoinositides. Recently, new phosphoinositide binders, such as PI(3,4,5)P₃-specific domains (Grp1), PI(4,5)P₂-specific domains (PLC 61), PI(4)P-specific domains (FAPP1), two PI(3)P specific binders (PEPP1 and AtPH1), two PI(3,4)P₂ specific binders (TAPP1 and TAPP2), have been reported. These particular phosphoinositide-binding domains can be engineered to be specific phosphoinositide and inositol phosphate recognition entities for both in vitro and cell-based assays. The Grp1 PH domain was employed in this application to be a model PI(3,4,5)P₃ binding reagent.

Assays that can measure the level of lipid kinase or phosphatase activity in tissues have the potential to become powerful research and diagnostic tools. In addition, assay platforms developed for measurement of phosphatase activity in research or clinical settings can be readily modified for use in in vitro assays for novel PI 3-Kinase, PTEN, SHIP, and other kinase/phosphatase inhibitors. Novel assays for detection of PI 3-Kinase, PTEN and SHIP activity disclosed in the present invention are useful for both determination of phosphoinositide kinase/phosphatase activity and identification of molecules which regulate the target enzyme activity.

### SUMMARY OF THE INVENTION

It has been recognized that it would be advantageous to develop homogenous HTS-compatible methods for detecting lipid kinase or phosphatase activity and the use thereof in disease detection and drug discovery. This application is a continuation-in-part of the above referenced co-pending U.S. patent applications Ser. Nos. 09/099,193 and 10/712,073, both of which are hereby fully incorporated by reference.

The present invention provides a lipid assay method which includes the step of first exposing a protein, having a lipid recognition motif that interacts with a target lipid and a competing lipid or inositol phosphate, to a solution containing the competing lipid. The method further includes the step of determining whether the target lipid is present in the solution. According to the method, the target lipid has a stronger affinity to the lipid recognition motif than does the competing lipid or inositol phosphate.

According to one embodiment of the invention, the assay determines activity of PI 3-Kinase, and the product lipid is PI(3,4,5)P₃. According to another embodiment of the invention, the assays detect lipid phosphatase activity using lipid or inositol phosphate substrates, which may be modified by the incorporation of a fluorescent molecule or other modifications, such as biotinylation. For example, the lipid phosphatase assayed in the present invention can be SHIP1, SHIP2, or PTEN, and the product lipid is PI(3,4)P₂ or PI(4,5)P₂, which is a de-phosphorylation product of a reaction between the lipid phosphatase SHIP1, SHIP2 or PTEN and the substrate lipid. The lipid phosphatase assays of the present invention can be used as a screening method for detection of a disease or a drug by detection of a predetermined level of the PI(3,4)P₂ or PI(4,5)P₂ product lipid.

The assay can be any of a number of assay types, but is preferably a plate-based assay. Examples include an enzyme linked immunosorbent assay (ELISA), an amplified luminescence proximity homogenous assay (ALPHA), and a fluorogenic assay such as fluorescence polarization (FP), fluorescence resonance energy transfer (FRET) or time-resolved fluorescence resonance energy transfer (TR-FRET). In the embodiment where the assay is an ELISA assay, prior to exposing the protein having a lipid recognition motif to a target lipid and a competing lipid or inositol phosphate, a substrate of the assay plate can be coated with the competing lipid. Preferably, the coating step includes coating a streptavidin-coated substrate with the competing lipid or inositol phosphate.

Additional competing and noncompeting lipids or inositol phosphates can also be present in the solution, enabling the assay method of the present invention to be used with complex solutions including bodily tissues, fluids, and plasma.

The present invention is further directed to a lipid assay kit, which includes a product lipid, a probe lipid or inositol phosphate, and a protein that has a lipid recognition motif that interacts with the product lipid and the probe lipid or inositol phosphate. The product lipid has a stronger affinity to the lipid recognition motif than the probe lipid or inositol phosphate. The assay kit can further include a multi-well assay plate. The multi-well assay plate preferably includes the probe lipid or inositol phosphate immobilized in the wells of the multi-well assay plate. The lipid assay kit can further include primary and secondary antibodies. As mentioned with respect to the assay method, additional competing and noncompeting lipids or inositol phosphates can also be present in the solution, enabling the assay method of the present invention to be used with complex solutions including bodily tissues, fluids, and plasma.

The present invention also provides methods for screening for a disease causing alteration of a lipid kinase or phosphatase by using the lipid kinase or phosphatase assay kit of the present invention to detect changes in the lipid phosphatase activity in bodily tissue, blood, or serum samples.

In addition, the present invention provides methods for screening for a compound having an enhancing or inhibiting effect on a lipid kinase or phosphatase using the lipid kinase or phosphatase assay method or the lipid kinase or phosphatase assay kit of the present invention to detect changes in the lipid kinase or phosphatase activity.

The present invention also provides a phosphoinositide fluorogenic assay which comprises a fluorescently labeled phosphoinositide probe; a fluorescence quencher-tagged lipid recognition protein (LRP) and the phosphoinositide analytes. This assay format is also suitable for detection of the activities of PI 3-Kinase, PTEN and SHIP. Two strategies were employed to attach the fluorescence quencher to the LRP. First, the LRP was covalently modified by a small molecule quencher. In the second approach, glutathione was modified with a small molecule quencher through its thiol-attachment and the glutathione-quencher conjugate formed a complex with the GST-tagged LRP.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

The accompanying drawings illustrate the present invention and are a part of the specification. Together with the following description, the drawings demonstrate and explain the principles of the present invention.

FIG. 1 shows a diagram of phosphoinositide interconversions, including the de-phosphorylation of PI(3,4,5)P₃ to PI(3,4)P₂ or PI(4,5)P₂ by SHIP 1, SHIP2, or PTEN, and the phosphorylation of PI(4,5)P₂ by PI 3-Kinase

FIG. 2 illustrates the principles of a competitive ELISA assay for lipid phosphatase activity.

FIG. 3 demonstrates the application of a competitive ELISA assay for the detection of PI(3,4)P₂ produced by SHIP2.

FIG. 4 shows the structures of synthetic phosphoinositides and inositol phosphates and modification via incorporation of fluorescent labels.

FIG. 5 shows the specificity of a competitive ELISA for detection of PI(3,4,5)P₃ versus PI(4,5)P₂, which can be applied for the detection of PI 3-Kinase activity

FIG. 6 shows the application of a competitive ELISA for detecting PI(3,4,5)P₃ produced by activation of cellular PI 3-Kinase in response to growth factor stimulation.

FIG. 7 shows the interaction of a PI(3,4,5)P₃ specific binding proteins with (A) FAM labeled PI(3,4,5)P₃, (B) TAMRA labeled PI(3,4,5)P₃ and I(1,3,4,5)P₄, and (C) BODIPY®-TMR labeled PI(3,4,5)P₃ and I(1,3,4,5)P₄ tracers as determined by fluorescence polarization.

FIG. 8 shows the interaction of a PI(3,4)P₂ specific LRP with fluorescently labeled PI(3,4)P₂ in a fluorescence polarization assay.

FIG. 9 shows the specific competition of PI(3,4,5)P₃ (closed squares) versus PI(4,5)P₂ (open squares) with a PI(3,4,5)P₃ specific LRP for interaction with (A) FAM-PI(3,4,5)P₃, (B) BODIPY®-TMR-PI(3,4,5)P₃, and (C) BODIPY®-TMR-I(1,3,4,5)P₄.

FIG. 10 shows the specific competition of PI(3,4)P₂ (closed squares) versus PI(3,4,5)P₃ (open squares) with a PI(3,4)P₂ specific LRP for interaction with fluorescently labeled PI(3,4)P₂ in a fluorescence polarization assay.

FIG. 11 shows determination of PI 3-Kinase activity using a competitive fluorescence polarization assay. Changes in signal are dependent on (A) reaction time and (B) enzyme concentration.

FIG. 12 shows determination of PI 3-Kinase activity using a competitive fluorescence polarization assay.

FIG. 13 shows the calculation of the Z′ value for a competitive fluorescence polarization assay for determination of PI 3-Kinase activity.

FIG. 14 shows the application of a competitive fluorescence polarization assay for PI 3-Kinase activity for determination of the pharmacological profile of the PI 3-Kinase inhibitors LY294002 (squares) and wortmannin (triangles).

FIG. 15 demonstrates the application of a competitive fluorescence polarization assay for detection of SHIP2 activity. Formation of product is dependent on (A) reaction time, and on (B) enzyme concentration.

FIG. 16 shows specific competition of PI(4,5)P₂ (circles) versus PI(3,4,5)P₃ (squares) with a PI(4,5)P₂ specific LRP in an AlphaScreen™ assay.

FIG. 17 shows specific competition of PI(3,4)P₂ (squares) versus PI(3,4,5)P₃ (triangles) with a PI(3,4)P₂ specific antibody in an AlphaScreen™ assay.

FIG. 18 shows the application of a competitive AlphaScreen™ assay for determination of PI 3-Kinase activity. Change in signal is dependent on the concentration of PI(4,5)P₂ substrate (A) and amount of PI 3-Kinase enzyme (B).

FIG. 19 shows the application of a competitive AlphaScreen™ assay for PI 3-Kinase activity for determination of the pharmacological profile of the PI 3-Kinase inhibitors LY294002 (circles) and wortmannin (squares).

FIG. 20 shows the calculation of the Z′ value for a competitive AlphaScreen™ assay for determination of PI 3-Kinase activity.

FIG. 21 shows the activity of the lipid phosphatases PTEN and SHIP2 against a variety of substrates, including synthetic phosphoinositides, inositol phosphates, biotinylated phosphoinositides, and fluorescently labeled phosphoinositides.

FIG. 22 illustrates the principles of fluorescence polarization assays for lipid phosphatase activity that incorporate the use of fluorescent substrates.

FIG. 23 illustrates the principles of FRET and TR-FRET assays for lipid phosphatase activity that incorporate the use of fluorescent substrates.

FIG. 24 shows the fluorescence quenching effects by small molecule quencher modified LRPs.

FIG. 25 illustrates a competitive dequenching assay using small molecule modified LRPs.

FIG. 26 shows detection of PI(3,4,5)P₃ generated from a PI 3-Kinase reaction using the fluorogenic system.

FIG. 27 shows the fluorescence quenching of BODIPY® FL-PI(3,4,5)P₃ by GS-QSY7.

FIG. 28 shows the fluorescence quenching effects of BODIPY® FL-PI(3,4,5)P₃ with increasing amounts of GST-Grp1.

FIG. 29 shows a competition fluorogenic assay with diC₁₆ PI(3,4,5)P₃.

FIG. 30 shows a fluorogenic competition with a mixture of diC₁₆ PI(4,5)P₂ and PI(3,4,5)P₃.

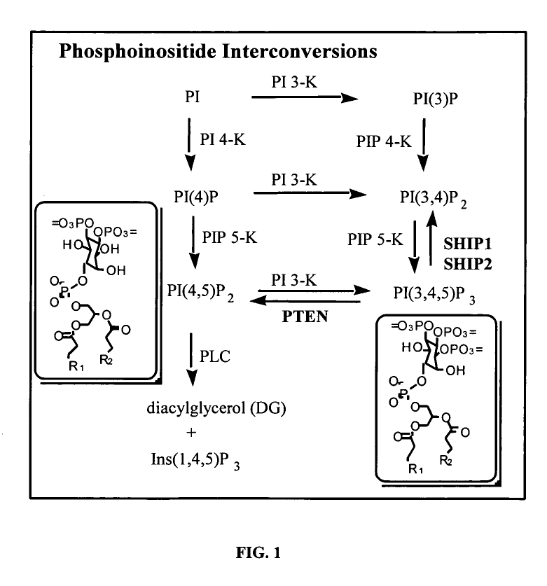

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

The present invention is directed to the use of lipid recognition proteins (LRPs) as detectors of the product lipid of a lipid phosphatase, in a convenient assay platform system that can be readily used in the industry.

The term “lipid phosphatase” refers to an enzyme which changes the phosphorylation state of its substrate, in this case a phosphoinositide lipid or an inositol phosphate, via removal of a phosphate group. Examples of such lipid phosphatases are SHIP1, SHIP2, PTEN, PTPRQ, SKIP, Myotubularin, MTMR2 and OCRL1.

The term “lipid kinase” refers to an enzyme which changes the phosphorylation state of its substrate, in this case a phosphoinositide lipid or an inositol phosphate, via addition of a phosphate group. Examples of such lipid kinases are PI 3-Kinases, PI 4-Kinases, and PI 5-Kinases.

LRPs that are used in accordance with the present invention can be recombinant proteins expressed as fusions of lipid recognition domains that are present in cellular proteins. The domains of the cellular proteins that interact with a lipid are fused to an affinity tag, such as glutathione-S-transferase (GST), myc, or FLAG, for example. The proteins having such domains are typically involved with such cellular functions as phosphorylation of lipids, or are adaptor proteins that assist in forming complexes with the cellular membrane to allow the cell membrane to interact with lipid structures. The domains from these proteins that allow them to perform such functions can be extracted from the naturally occurring proteins, or prepared through recombinant methods, and can then be fused to GST to form an LRP.

The pleckstrin homology(PH)domain is well known to those skilled in the art. Furthermore, different PH domains often exhibit specificity for different phosphoinositides. See Dowler et al., 2000 Biochem J. 351:19-31, which is incorporated herein by reference. All PH domains are predicted to fold into a similar 3-dimensional structure, and may mediate protein-lipid interactions, protein-protein interactions, or both. Polypeptides with PH domains of determined tertiary structure include pleckstrin, spectrin, dynamin, and phospholipase Cγ. Gray et al. (Anal. Biochem. 313:234-245. (2003)) describe the use of a PH domain derived from the Grp1 protein (General Receptor for Phosphoinositides) in assays designed to detect loss of PI(3,4,5)P₃ substrate as a method for assaying lipid phosphatase activity. The assays described here differ in principle in that they utilize lipid-specific antibodies or LRPs to specifically detect the formation of the product of the enzymatic reaction. Assays by measuring the formation of the reaction products results are more sensitive assays.

In a broad sense, the present invention involves methods, assay kits and apparatuses that use a specific LRP as a detection reagent for specific lipids or inositol phosphates in an enzyme assay for lipid metabolism. More particularly, the present invention involves the use of a lipid recognition protein (LRP) as a probe that interacts specifically with a product lipid or inositol phosphate of a lipid phosphatase in such assays. For example, the phospholipase Cδ (PLCδ) and TAPP proteins both include PH domains. While their PH domains vary in the specificity of their interactions with various phosphoinositides, the PLCδ PH domain exhibits a strong preference for interaction with PI(4,5)P₂ and the TAPP PH domains exhibit a strong preference for PI(3,4)P₂. Thus, in a narrow sense, the present invention involves methods that use a lipid detector protein having a binding specificity for a target lipid of a lipid kinase or phosphatase as a lipid detection probe to measure the target lipid of a lipid kinase or phosphatase. Therefore, the minimal components necessary for a phosphoinositide phosphatase or kinase assay of the present invention are 1) a source of enzyme, 2) the appropriate phosphoinositide substrate, and 3) a detection reagent (either a LRP or antibody) with specificity for the phosphoinositide or inositol phosphate product of the enzymatic reaction.

For assays of phosphatase or kinase activity in cells or tissues, PI 3-Kinase, PTEN or SHIP can be isolated by immunoprecipitation using specific antibodies and protein A-agarose. Antibodies suitable for isolation of PI 3-Kinase, PTEN or SHIP are available from several commercial sources, including Upstate Biotechnology (NY) and Santa Cruz Biotechnology (CA). For the purposes of assay application to HTS and drug discovery, a source of recombinant enzyme is preferable, i.e., vectors for recombinant expression of affinity tagged enzymes in bacterial or insect cell culture systems. Other sources of phosphoinositide phosphatase or kinase expression systems are also suitable.

A number of options are available for obtaining enzyme substrate mixtures. Both PTEN and SHIP use PI(3,4,5)P₃ as their preferred substrate, while type I PI 3-Kinases use PI(4,5)P₂ as their preferred substrate. Synthetic short-chain PI(4,5)P₂ and PI(3,4,5)P₃ (diC₄, diC₈) has the advantage of being soluble in aqueous solution without requiring incorporation into liposomes or micelles. The substrate may also be present in the form of long chain synthetic phosphoinositides, such as diC₁₆ PI(4,5)P₂ or PI(3,4,5)P₃, incorporated into liposomes or micelles. This requires the mixing of long acyl-chain (diC₁₆ to diC₂₄) PI(3,4,5)P₃ with the appropriate ratios of carrier lipids, such as phosphatidylcholine or phosphatidylethanolamine, in an organic solvent. Following evaporation of the solvent to produce a lipid film, water or a suitable buffer solution is added to rehydrate the lipids. Micelles may be prepared by sonication of the mixture, while passage through an extruder is necessary to produce a homogenous population of bilaminar liposomes.

Soluble diC₄ or diC₈ PI(3,4,5)P₃ is the straightforward choice as substrate in an in vitro assay. It has been shown that the diC₄ and diC₈ synthetic phosphoinositides are suitable as substrates. In addition, a fluorescently-labeled diC₆ PI(3,4,5)P₃ has already been shown to act as a substrate for PTEN. While it is necessary to test each enzyme to determine which method of substrate presentation is optimal, previous results demonstrate that soluble diC₄ and diC₈ phosphoinositides are suitable substrates for use in lipid phosphatase and kinase assays.

One embodiment of the present invention is an Enzyme Linked Immunosorbent Assay (ELISA) to determine activity of a lipid phosphatase or kinase. In order to readily distinguish PI(4,5)P₂ from other phosphoinositides, a derivative such as biotinylated diC₆ PI(4,5)P₂ or I(3,4,5)P₃ is immobilized in the wells of streptavidin-coated assay plates. As an example, a 96-well plate can be used, although the present embodiment of the invention is clearly adaptable for a variety of assay plate sizes and formats. Initial experiments establish the range of LRP detector protein and biotinylated diC₆ PI(4,5)P₂ or I(3,4,5)P₃ concentration where detection of the target phosphoinositide is optimized in an assay tray format. For example, in a standard curve binding procedure, a 96-well streptavidin-coated assay plate marketed as StreptaWell® (Roche) is coated with increasing amounts of biotinylated PI(4,5)P₂ per well. The coated wells are then blocked for an hour at room temperature using 100 μL of Stabilguard® (SurMedics) per well. The samples are then incubated with 10 pmol of GST-tagged LRP in a 100 μL volume per well. Several washes are then performed. Next, 100 μL of a 1:1000 dilution of an anti-GST HRP-conjugated antibody, provided by Sigma, is added to each well. The GST HRP-conjugated antibody is provided as a reagent that interacts with the GST portion of the lipid recognition protein, and hence allows for subsequent colorimetric detection. After one hour of incubation at room temperature, the plates are washed and 100 μL of 3′,3′,5′,5′-tetramethylbenzidine (TMB) substrate solution as a development reagent (Sigma) is added to each well. Following color development, the reaction is stopped by the addition of 100 μL 0.5 M H₂SO₄ and the absorbance at 450 nm is measured. The results of the absorbance measurements represent the binding of the LRP detector protein to increasing amounts of immobilized diC₆ PI(4,5)P₂. A competition procedure can be performed using similar methodology. Assay plates such as the StreptaWell® microplates, used in the previously discussed optimization process, are prepared by coating the wells with 10 pmol of biotinylated diC₆ PI(4,5)P₂ per well. In a separate incubation apparatus, 10 pmol of LRP is preincubated with increasing amounts of closely related derivative phosphoinositides. For example, the LRP is preincubated with either diC₈ PI(4,5)P₂ or diC₈ PI(3,4,5)P₃, prior to binding to the biotinylated diC₆ PI(4,5)P₂ coated surface of the assay plate wells.

The results of the competition procedure can be determined by, for example, measuring absorbance (450 nm) for both PI(4,5)P₂ and PI(3,4,5)P₃ at various pmol increments of competing phosphoinositide or inositol phosphate. The difference in competitiveness is particularly evident at lower levels of phosphoinositides, and the difference will clearly exemplify the ease with which the assay can distinguish between the two phosphoinositides. An ELISA assay for detection of the products of phosphoinositide phosphatase or kinase activity may be designed as either a direct or a competitive assay.

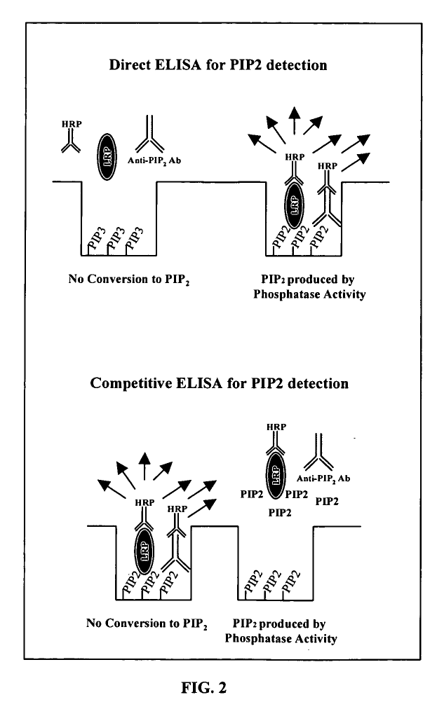

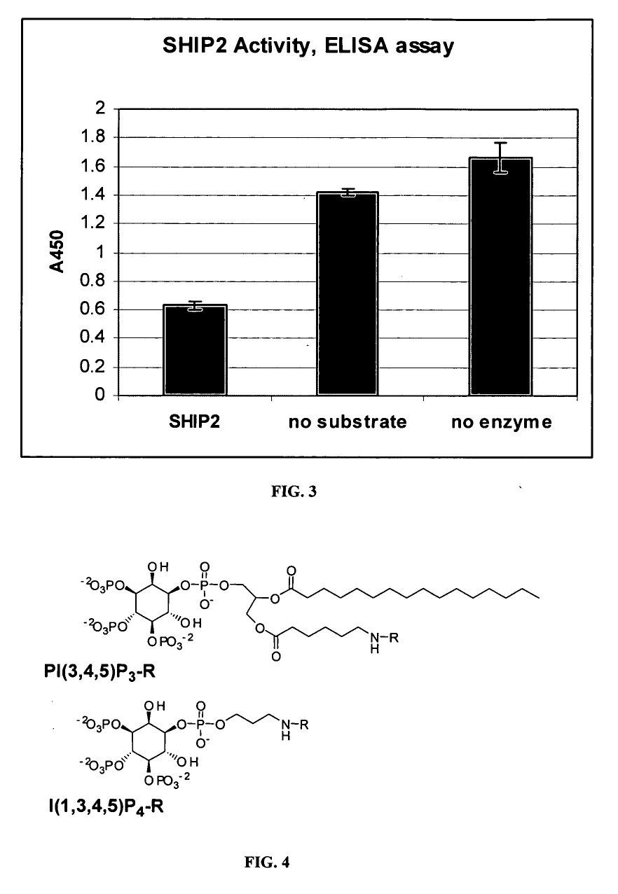

FIG. 2 illustrates the principles of a competitive ELISA for lipid phosphatase activity. Components of this assay are a plate coated with the product produced by phosphatase activity, in this case, PI(4,5)P₂, a lipid recognition protein or antibody that specifically interacts with this product but does not interact significantly with the substrate phosphoinositide, in this case, PI(3,4,5)P₃. In this example, PI(4,5)P₂ is produced by the activity of a lipid phosphatase, such as PTEN. In the absence of enzyme activity, the protein or antibody binds to the coated plate. In the presence of enzyme product, binding of the protein or antibody is competed for, and the amount of binding decreases as the amount of product increases. Secondary detection is accomplished using an appropriate secondary antibody conjugated to horseradish peroxidase or alkaline phosphatase, followed by colorimetric development using a substrate such as TMB. The use of fluorescently labeled secondary antibodies is also a possibility. In this type of competitive assay, the signal is inversely proportional to the amount of enzyme product produced in the reaction. FIG. 3 shows the application of a competitive ELISA for the detection of SHIP2 conversion of PI(3,4,5)P₃ to PI(3,4)P₂.

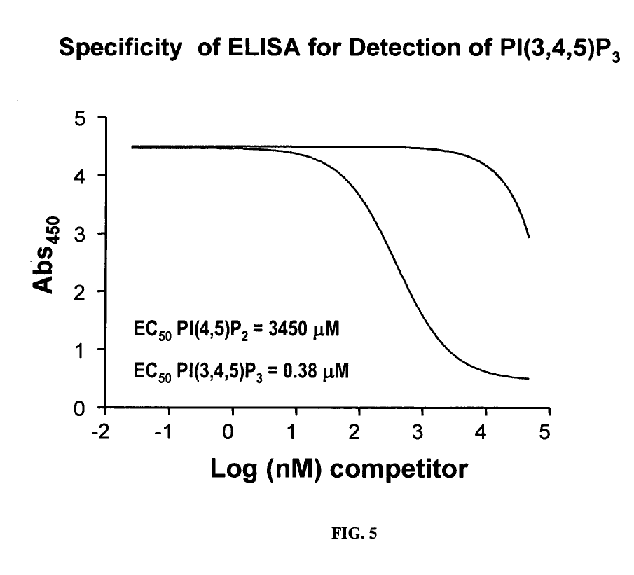

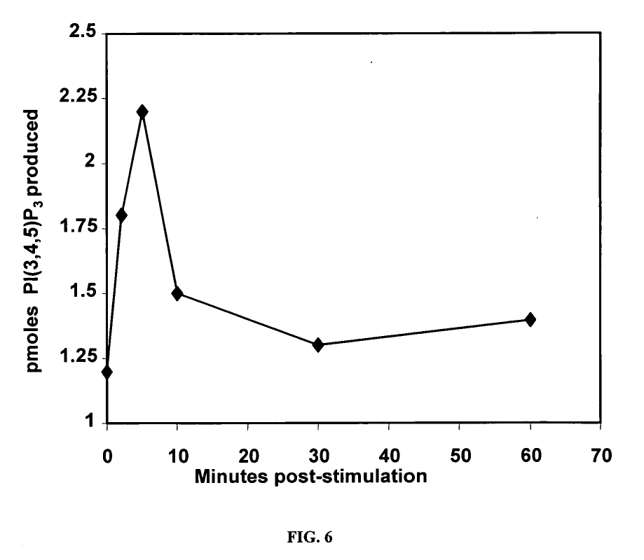

A similar competitive ELISA can be used to detect PI 3-Kinase activity using similar principles. Enzyme is incubated with diC₈ PI(4,5)P₂ substrate for an appropriate period of time. The enzyme reaction mixture is then mixed with a solution of PI(3,4,5)P₃-specific LRP or antibody and incubated for an hour. This mixture is added to the wells of a PI(3,4,5)P₃ coated plate, followed by several wash steps and detection using the appropriate secondary antibody, such as a HRP-conjugated anti-GST antibody or an HRP-conjugated anti-IgG antibody, followed by colorimetric detection. The PI(3,4,5)P₃ present in the reaction mixture competes with the LRP or antibody for binding to the plate so that absorbance is inversely proportional to enzyme activity. This type of competitive ELISA can be applied for the detection of PI 3-Kinase activity following growth factor stimulation of cells. PI 3-Kinase was isolated from cell lysates at several time points following growth factor stimulation. The enzyme was isolated via immunoprecipitation using an antibody against the p85 subunit of the enzyme, which is available from several commercial sources, and protein A agarose, also commonly available. The enzyme was incubated with 5 μM of diC₈ PI(4,5)P₂, and then a competitive ELISA assay was used to detect generation of PI(3,4,5)P₃. FIG. 5 shows the time course of PI 3-Kinase activation following growth factor stimulation obtained using this method. Picomoles of PI(3,4,5)P₃ produced were determined by comparison to a standard curve.

In fluorescence polarization assays, light from a monochromatic source passes through a vertical polarizing filter to excite fluorescent molecules in a sample tube or microplate well. Only those molecules that are oriented in the vertically polarized plane absorb light, become excited, and subsequently emit light. The intensity of the emitted light is measured both parallel and perpendicular to the excitation light. The fraction of the original, incident, vertical light intensity that is emitted in the horizontal plane indicates the amount of rotation that the fluorescently labeled molecule has undergone while in the excited state, and is a measure of its relative size. Changes in the relative size of a fluorescently labeled molecule may be due to interactions with another molecule, dissociation, enzymatic modification, degradation, or conformational change.

In order to use these reagents in a competitive FP assay, unlabeled phosphoinositide products of an enzymatic reaction must successfully compete with the labeled tracer for interaction with the binding proteins. In addition, the level of cross-reactivity of binding proteins with the phosphoinositides used as enzyme substrates should be minimal. The FP assays developed for the detection of PI 3-Kinase and SHIP2 activity were designed as competitive assays in which the enzyme product would compete with a fluorescently labeled synthetic phosphoinositide or inositol phosphate tracer for binding to a specific phosphoinositide binding protein. As the products of the enzymatic reaction increase, the soluble phosphoinositide products compete for the interaction of the binding protein and the fluorescent tracer, leading to a decrease in mP values as the amount of depolarized free tracer increases. Thus, changes in polarization should be inversely proportional to the degree of enzyme activity.

These assays involve four steps. First, the enzyme and substrate phosphoinositide are combined in an appropriate reaction buffer and the reaction is allowed to proceed. The use of water soluble synthetic diC₈-phosphoinositides as the enzyme substrate eliminates the need for liposome formation and results in more consistent assay conditions. Following the incubation, the reactions are quenched by the addition of a chelator. A mixture of phosphoinositide binding protein is added and mixed, followed by addition of a fluorophore-labeled phosphoinositide tracer. Finally, polarization values are measured to determine the extent of enzyme activity in the reaction.

Unlike previously developed assays for phosphoinositide kinase and phosphatase activity, that simply detect the incorporation or release of a phosphate group, these assays are designed to detect the formation of a specific phosphoinositide product. In the case of assays for PI 3-Kinase and SHIP2 activity these products are PI(3,4,5)P₃ and PI(3,4)P₂, respectively. GST-tagged phosphoinositide-specific binding proteins that have been previously characterized were evaluated for binding to fluorophore labeled phosphoinositide and inositol phosphate tracers. The PH domain of GRP1 (general receptor for phosphoinositides) was used as a detector for PI(3,4,5)P₃ and GST-TAPP1 (tandem PH-domain-containing protein) was used as a detector for PI(3,4)P₂.

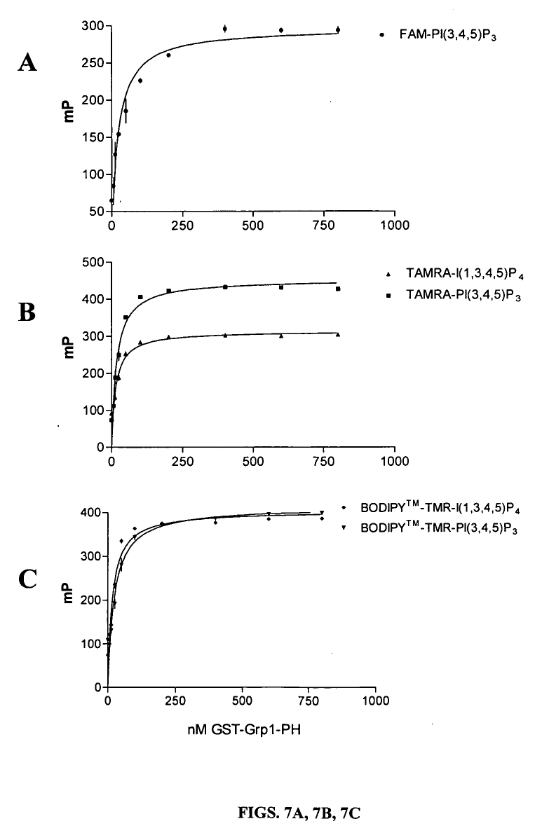

Initially, a variety of fluorescently-tagged synthetic phosphoinositide and inositol phosphates were evaluated as tracers for detection of PI(3,4,5)P₃. Because the interaction of PH domain containing proteins with phosphoinositides occurs chiefly through binding to the inositol head groups of these lipids, labeled inositol phosphates were included in this evaluation. FAM, BODIPY®-TMR, and TAMRA labeled tracers were evaluated for binding to GST-GRP1-PH. Tracer concentrations were held at 10 nM and increasing amounts of protein were added to determine the concentration required for maximal capture. The binding curves for FAM-PI(3,4,5)P₃, TAMRA-I(1,3,4,5)P₄ BODIPY®-TMR-PI(3,4,5)P₃ are shown in FIGS. 7A, 7B and 7C respectively. In most cases, greater maximal binding and affinity were observed for the interaction of the GRP1 PH domain with TAMRA and BODIPY®-TMR labeled tracers when compared to FAM-PI(3,4,5)P₃. The change in polarization values produced by protein interaction with the red fluorophore labeled tracers was also considerably higher. Table 1 lists the Bₘₐₓ and K_{d} values for different binding protein/tracer combinations at 10 nM tracer concentrations.

TABLE 1
Bₘₐₓ and K_{d} values for binding of GST-GRP1-PH
to fluorescent phosphoinositides
and inositol phosphates.

| Tracer | Bₘₐₓ (mP) | K_{d} (nM) |
|---|---|---|
| FAM-PI(3,4,5)P₃ | 296 | 21.7 |
| BODIPY ®-TMR-PI(3,4,5)P₃ | 412 | 23.6 |
| BODIPY ®-TMR-I(1,3,4,5)P₄ | 403 | 16.5 |
| TAMRA-PI(3,4,5)P₃ | 452 | 17.4 |
| TAMRA-I(1,3,4,5)P₄ | 313 | 14.2 |

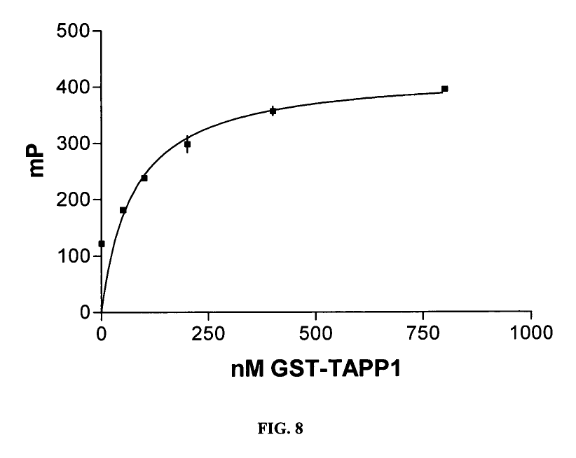

The interaction of recombinant GST-TAPP1, a PI(3,4)P₂ specific binding protein, with BODIPY®-FL and BODIPY®-TMR labeled PI(3,4)P₂ was also compared with similar results. The Bₘₐₓ and K_{d} values for binding of GST-TAPP1 to a BODIPY®-TMR -PI(3,4)P₂ tracer at 5 nM were 425 mP and 75 nM respectively (FIG. 8).

In order to use these reagents in a competitive FP assay, unlabeled phosphoinositide products of an enzymatic reaction must be able to successfully compete with the labeled tracer for interaction with the binding proteins. In addition, the level of cross-reactivity of binding proteins with the phosphoinositides used as enzyme substrates should be minimal. To examine the suitability of these phosphoinositide binding proteins for this application, competition assays were performed using optimal amounts of lipid binding proteins and fluorescent tracers, mixed with increasing amounts of diC₈-phosphoinositide competitors.

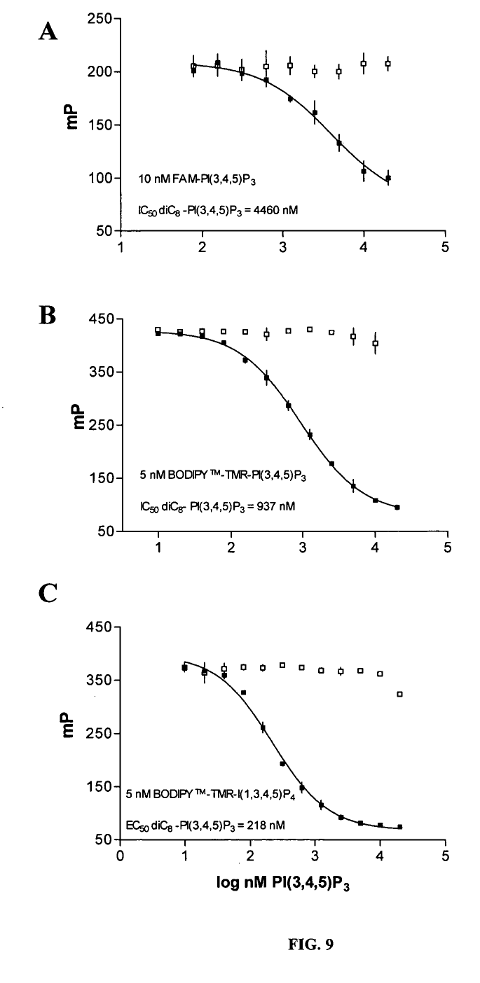

The sensitivity of this approach for detecting conversion of PI(4,5)P₂ to PI(3,4,5)P₃ was evaluated using GST-GRP1-PH protein and FAM and BODIPY®-TMR labeled tracers. Competition assays in which both diC₈-PI(3,4,5)P₃ and diC₈-PI(4,5)P₂ were added at increasing concentrations were performed. In all cases, the interaction of the binding protein with fluorescent tracers was effectively displaced by increasing PI(3,4,5)P₃, while the presence of PI(4,5)P₂ in amounts over 100-fold greater had a minimal effect (FIG. 9). This high degree of selectivity indicates that potential interference due to cross-reactivity of the GST-GRP1-PH with PI(4,5)P₂ substrate in a PI 3-Kinase assay will not be a factor in assay performance. Experiments using BODIPY®-TMR labeled tracers showed greater sensitivity, in part because these could be used at a lower concentration (5 nM) than the FAM-labeled tracer (10 nM) with satisfactory results. BODIPY®-TMR-I(1,3,4,5)P₄ tracer showed the highest degree of sensitivity for displacement by diC₈-P(3,4,5)P₃, with an IC₅₀ of 218 nM, and was chosen for continuing work in PI 3-Kinase assay development.

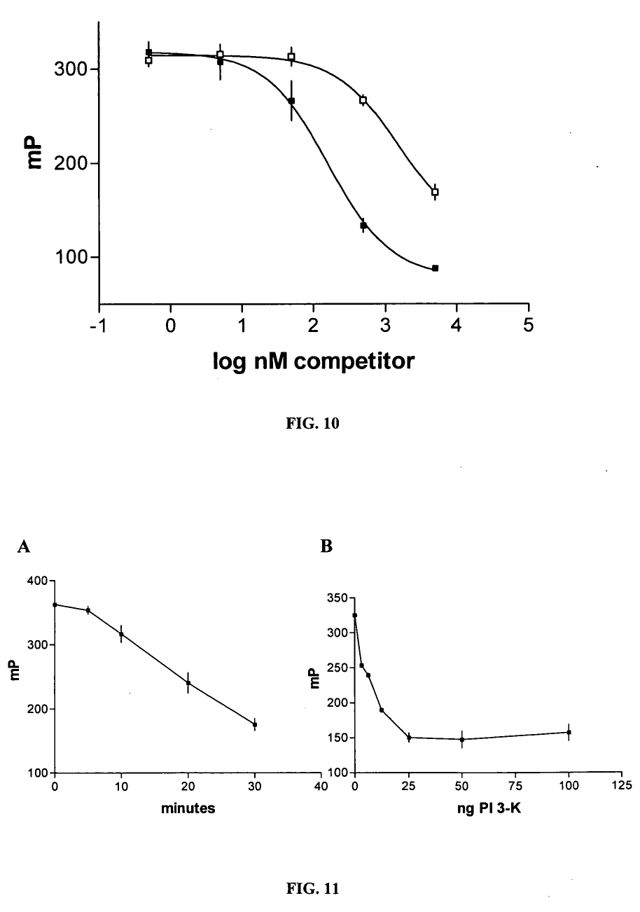

Similar competition assays were performed to examine the selectivity and sensitivity of PI(3,4)P₂ detection by GST-TAPP1 (FIG. 10). In these assays, the concentrations of GST-TAPP1 and the fluorescently labeled phosphoinositide were held constant as the concentration of unlabeled lipid competitor was increased. Protein binding to a BODIPY®-TMR-PI(3,4)P₂ tracer was displaced by diC₈-PI(3,4)P₂ with an IC₅₀ value of 165 nM and by diC₈-PI(3,4,5)P₃ with a predicted IC₅₀ of 1508 nM. While this assay is significantly less selective, the degree of selectivity should be sufficient for designing a SHIP2 assay. In implementing these assays, the initial amount of PI(3,4,5)P₃ substrate should be kept low enough to minimize competition.

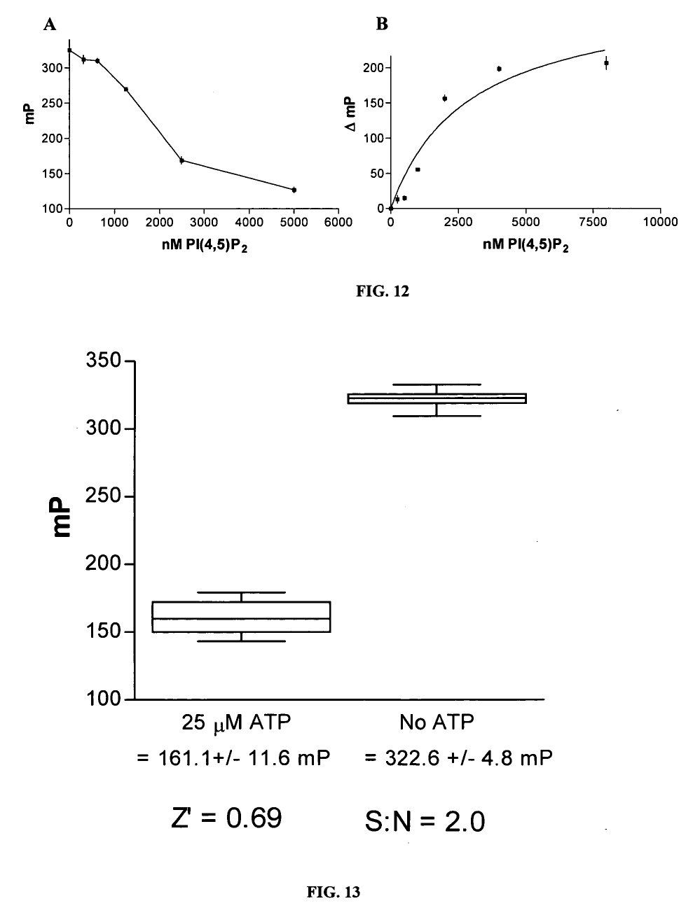

The ability of FP competitive assays to detect the production of phosphoinositide products of PI 3-Kinaseα activity was tested using recombinant PI 3-Kinaseα. Increasing amounts of enzyme were added to diC₈-PI(4,5)P₂ substrate. The production of PI(3,4,5)P₃ was detected as a decrease in mP values as enzyme products compete with I(1,3,4,5)P₄-BODIPY®-TMR for interaction with the binding protein. PI(3,4,5)P₃ production was dependent on reaction time and on enzyme concentration. (FIG. 11). At constant enzyme levels, polarization values decreased with increasing amounts of substrate, and substrate conversion exhibited saturation kinetics with a Kₘ of approximately 3 μM (FIG. 12 A).

The suitability of these assays for HTS applications was evaluated by determining the signal to noise ratio and Z′ factor, a measurement of assay robustness and suitability for adaptation to automated applications, for 50 replicates of typical PI 3-Kinase enzyme reactions and mock reactions in which no ATP was added. While the signal to noise ratio is low, typical of many FP based assays, the Z′ factor was determined to be 0.69 (FIG. 13). Z′ values of greater than 0.5 are considered to be an indication that the assay is robust enough for HTS applications (Zhang et al., J. Biomol. Screening, 4:67-73. (1999)). Assay performance was unaffected by DMSO levels of up to 5% and mP values were stable for up to six hours, the longest time period tested.

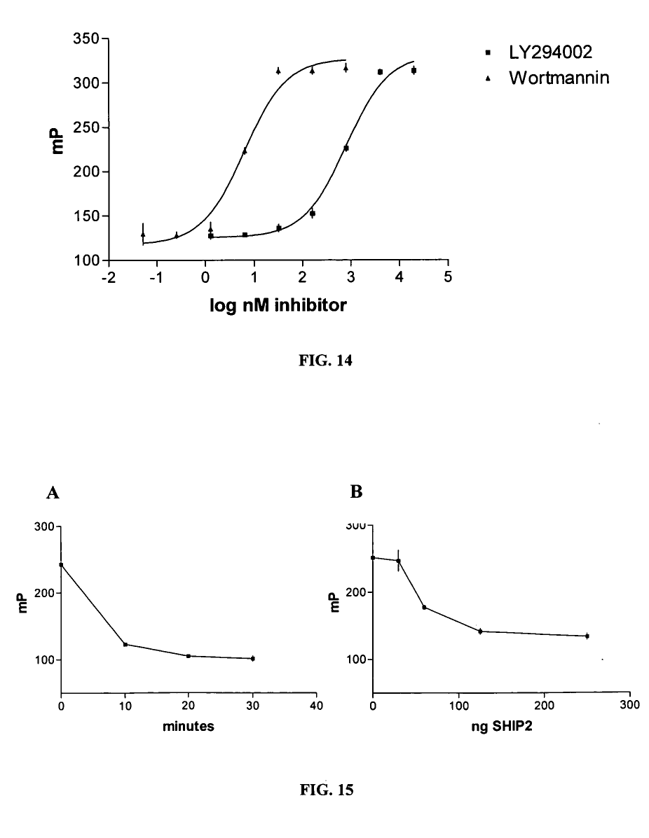

The potential for these assays for application to the detection and characterization of enzyme inhibition was determined using the well-characterized PI 3-Kinase inhibitors wortmannin and LY294002 (FIG. 14). IC₅₀ values for LY294002 and wortmannin were determined to be approximately 800 nM and 5 nM, respectively. Table 2 compares the IC₅₀ values obtained in this study with those determined using several other approaches, including radiometric and homogeneous assays.

TABLE 2
Comparison of IC₅₀ values determined for PI 3-K inhibitors
with published values.

| | | IC₅₀ for PI 3-Kα |
|---|---|---|
| Compound | Reported IC₅₀ Values | using FP assay |
| LY294002 | 1.4 μM, PI 3-K from bovine brain | 0.8 μM |
| | 2.8 μM, recombinant PI 3-Kγ | |
| | 5.6 μM, PI 3-Kγ | |
| Wortmannin | 1.9 nM, PI 3-K from bovine brain | 5 nM |
| | 4.2 nM, PI 3-K from bovine brain | |
| | 1.45 nM, recombinant PI 3-Kγ | |
| | 12 nM, PI 3-Kγ | |

A similar assay, designed to detect the production of the phosphoinositide products of SHIP2 activity using GST-TAPP1, was also evaluated. Increasing amounts of SHIP2 enzyme was added to reactions containing diC₈-PI(3,4,5)P₃ substrate. The production of PI(3,4)P₂ was detected as a decrease in mP values as enzyme products compete with PI(3,4)P₂-BODIPY®-TMR for interaction with the binding protein. PI(3,4)P₂ production was dependent on reaction time and on enzyme concentration (FIG. 15). Characteristics of this assay were similar to those of the PI 3-Kinase assay, including a Z′ value greater than 0.5, lack of interference by DMSO, and mP value stability over time. There are currently no known inhibitors of SHIP2 or any other phosphoinositide phosphatases that are available for further validation of assay performance.

One clear advantage of the FP assays described here is lack of requirement for additional assay components, such as the donor and acceptor beads which are used in AlphaScreen™ assays, in addition to the protein and lipids. FP approaches also require that only the lipid component of the assay be labeled with a fluorescent tag, in contrast to FRET and HTRF methods, which require multiple labeling reactions. Fluorophores are readily incorporated into synthetic phosphoinositides and inositol phosphates as a final step in their synthesis. Our initial evaluation of several different dye-labeled tracers suggests that a wide variety of dyes can be used as labels without compromising interactions with specific protein detectors. FP measurements are ratiometric rather than intensity-based, which leads to several advantages over other fluorescent assay approaches in which values are based on signal intensity, including greater ease of miniaturization and less susceptibility to artifacts produced by exogenous fluorescence or quenching from library compounds. The probability of these types of interference is even further reduced by the use of red-shifted tracers, such as those used in the PI 3-Kinase and SHIP2 assays presented herein.

The competitive FP assays of the present invention for the detection of phosphoinositide kinase and phosphatase activity are sensitive, robust, and suitable for adaptation to HTS formats. The approaches described herein can also be used as assays targeted at other lipid modifying enzymes, using suitable lipid binding proteins or antibodies and fluorophore-labeled lipids. Given the importance of lipid kinases, phosphatases, and phospholipases in human pathologies, these methodologies should prove very valuable for drug discovery.

The principles of the present invention can also be applied to other assay methods, such as the type using ALPHAScreen® reagents and the Fusion Alpha Universal Microplate Analyzer from PerkinElmer Life Sciences. The ALPHAScreen™ system detects emission shifts due to reactions involving the transfer of singlet oxygen. More particularly, the system uses photosensitive donor beads which convert ambient oxygen to a singlet state upon illumination at 680 nm. If an acceptor bead is in close proximity to the donor bead, due to a biological interaction, the diffusion of singlet oxygen activates chemiluminscent receptors and fluorescent acceptor molecules on the bead, resulting in an emission shift from 520 to 620 nm.

The ALPHAScreen™ assay format is an example of yet another assay method that can be used in accordance with the principles of the present invention. The ALPHAScreen™ format has the advantages of high specificity and sensitivity, and requires significantly less protein and lipid reagents than the ELISA assay format.

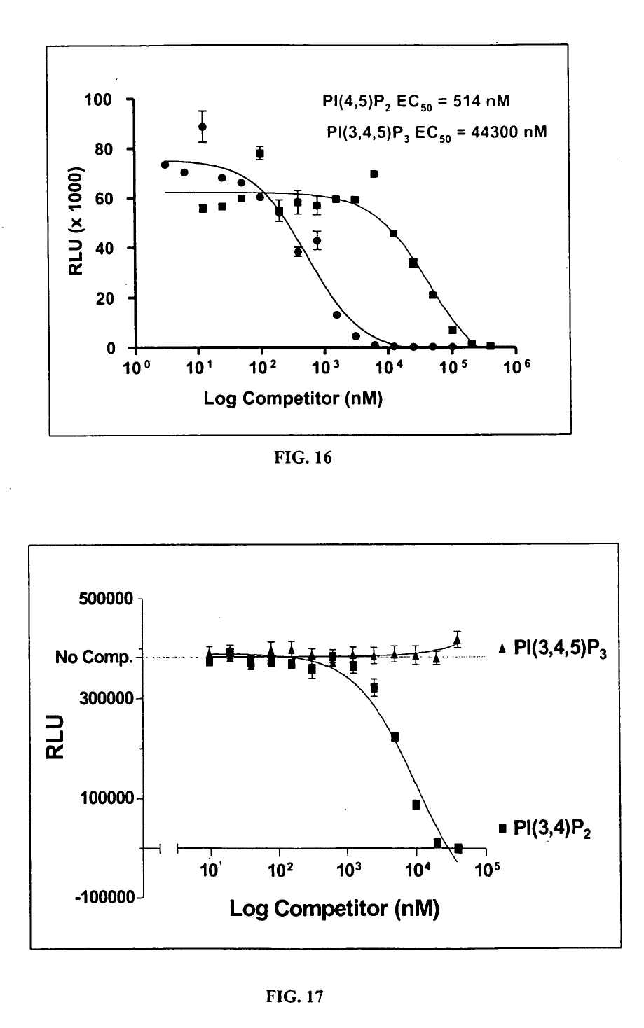

The present invention provides ALPHAScreen™ assays to detect specific binding and competition of phosphoinositide diphosphates using antibodies or LRPs. This approach can be used in competitive assays for the detection of PI(3,4)P₂ and PI(4,5)P₂ produced by lipid phosphatase or kinase activity. FIG. 16 shows the results of a binding and competition experiment using a PI(4,5)P₂-specific LRP derived from PLCδ and biotinylated PI(4,5)P₂. Increasing amounts of unlabeled PI(4,5)P₂ or PI(3,4,5)P₃ were added while levels of PLCδ and PI(4,5)P₂ were kept constant. PI(4,5)P₂ competition is approximately 90-fold greater than PI(3,4,5)P₃ competition, indicating a high degree of specificity for recognition of PI(4,5)P₂. FIG. 17 shows a similar competition experiment using anti-PI(3,4)P₂ monoclonal antibodies, described previously. The antibody shows a high degree of specificity for PI(3,4)P₂ versus PI(3,4,5)P₃.

The ALPHAScreen™ assay may be implemented as a competitive assay for the detection of PI 3-Kinase, PTEN or SHIP activity. The appropriate lipid substrate will be incubated with enzyme, and the resulting lipid mixture will be added to ALPHAScreen™ donor and acceptor beads. For example, for detection of PI(4,5)P₂ production by PTEN, components of the reaction mixture will be streptavidin-coated donor beads, biotinylated PI(4,5)P₂ or I(1,4,5)P₃, a PI(4,5)P₂-specific GST-tagged LRP or IgG antibody and either anti-IgG or anti-GST acceptor beads. An assay designed to detect PI(3,4)P₂ produced by SHIP would contain protein detection reagents or antibodies with specificity for that phosphoinositide product.

An assay designed to detect PI(3,4,5)P₃ produced by PI 3-Kinase would contain protein detection reagents or antibodies with specificity for that phosphoinositide product. Components would include biotinylated diC₆ PI(3,4,5)P₃, a PI(3,4,5)P₃ specific LRP or antibody, and diC₈ PI(4,5)P₂ substrate. These would be combined with Streptavidin-conjugated donor beads and anti-GST or anti-IgG conjugated acceptor beads from PerkinElmer. The assay can be performed directly in the wells of a 384-well plate as follows: 2.5 microliters of kinase buffer, which may contain compounds to be tested as PI 3-Kinase inhibitors, are added per well, 5 microliters of enzyme diluted in a kinase buffer is added, then 2.5 microliters of PI(4,5)P₂ substrate is added. The enzyme reaction is allowed to proceed. Five microliters of 10 nM biotinylated PI(3,4,5)P₃ in detection buffer is added, followed by 5 microliters of 10 nM PI(3,4,5)P₃-specific LRP, and 5 microliters of a 100 microgram/mL solution of ALPHAScreen™ donor and acceptor beads. The plate is incubated at 23° C. in the dark, and then read on a plate reader equipped for ALPHAScreen™ detection. The signal is inversely proportional to the amount of PI(3,4,5)P₃ product produced.

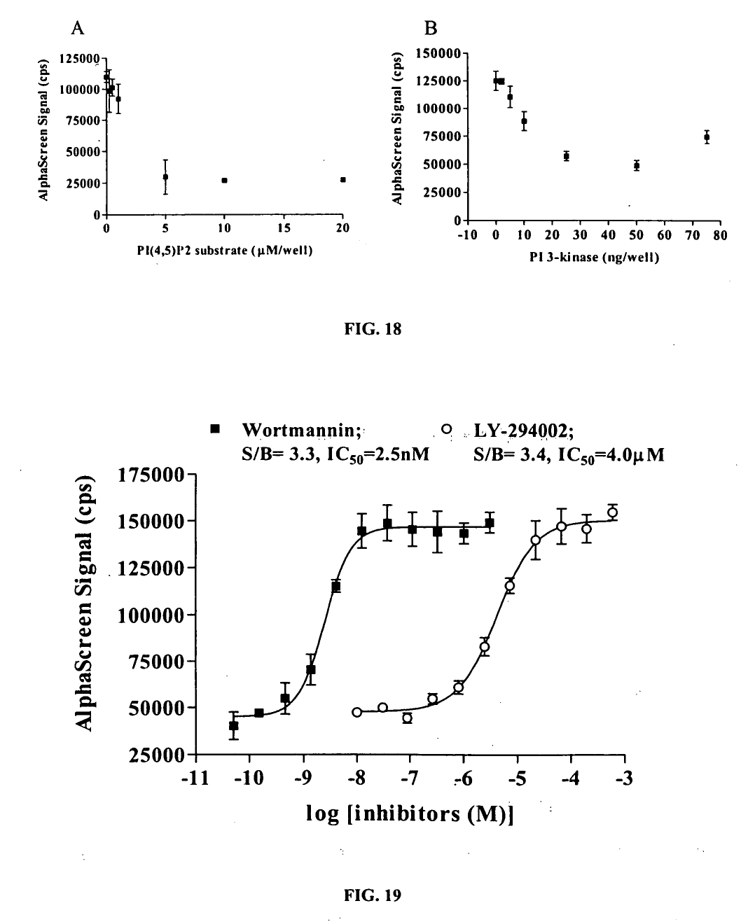

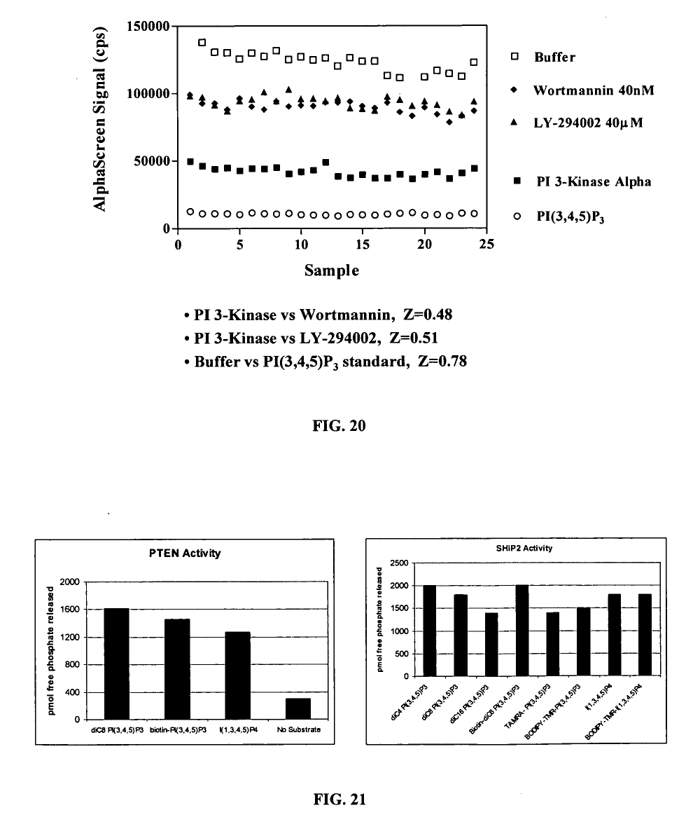

FIG. 18 shows the application of such an ALPHAScreen™ assay for the detection of PI(3,4,5)P₃ produced by PI 3-Kinase activity, using PI 3-Kinase alpha. The amount of product produced is both substrate and enzyme dependent. Similar results are obtained using PI 3-Kinase gamma, and the assay is suitable for other PI 3-Kinase isoforms. FIG. 19 shows the application of the assay for determination of the IC₅₀ values of known PI 3-Kinase inhibitors LY294002 and wortmannin. FIG. 20 shows assay precision as determined by Z′ using these inhibitors. The assay is robust, selective for detection of PI(3,4,5)P₃, displays expected pharmacological profiles for known PI 3-Kinase inhibitors, and shows acceptable precision for use in HTS.

Furthermore, the principles of the present invention can be readily applied to other assay formats for detection of lipids or associated enzyme activity using the LRPs or lipid-specific antibodies as detection devices. This includes homogeneous fluorescence methodology, in particular FP, FRET, and TR-FRET. In “direct” fluorescence-based assays, enzyme alteration of a fluorophore-labeled lipid substrate produces a change in interaction with an LRP or antibody. A similar type of direct assay for detection of protein kinase activity using fluorescent substrates has been developed and marketed by Molecular Devices (Sunnyvale, Calif.) (Gaudet, et al., J. Biomol. Screening, 8:164-175. (2003), Sportsman, et al., Comb. Chem. High Throughput Screening, 6:195-200. (2003)). In addition to competitive assays, it may also be advantageous to develop assays in which enzymes act directly upon a fluorescently labeled lipid substrate to produce a change in its interaction with a detector protein or antibody.

Fluorophore-labeled diC₆ phosphoinositides and P-1 aminopropyl-modified inositol phosphates can be synthesized. For example, dyes can be covalently attached to the end of one of the acyl chains for the phosphoinositides and at the P1 phosphate for the inositol phosphates, and are readily incorporated into synthetic phosphoinositides and inositol phosphates as the final synthetic steps. Sample structures of fluorophore labeled phosphoinositide and inositol phosphate tracers are shown for PI(3,4,5)P₃ and 1(1,3,4,5) P₄ (FIG. 4). A wide variety of dyes can be used as labels in FP assays without compromising interactions with specific protein detectors. It has been shown that SHIP2 has activity against fluorophore-labeled phosphoinositide substrates, and published work on PTEN indicates that it has similar activity(Maehama, et al., Anal Biochem, 279:248-50. (2000)). Further studies of enzyme activity toward and preference for different fluorescent substrates can be easily done using the malachite green assay for phosphate release. FIG. 21 shows determination of the phosphatase activity of PTEN and SHIP2 toward selected synthetic phosphoinositide and inositol phosphate substrates. A second requirement is that the detector proteins used must show sufficient specificity for the detection of the fluorescent phosphoinositide product versus the fluorescent phosphoinositide substrate. For PTEN and SHIP assays, PLCδ and TAPP1 are the preferred LRPs respectively.

Other major variables to address are the concentration of fluorescent substrates in the enzymatic reaction, the final concentration of fluorescent phosphoinositides in the assay read step, and the amount of detector protein required for optimal signal and sensitivity. Other than optimal substrate concentration, these variables will need to be addressed separately for FP and FRET assays. Approach used for FP assays can be modified appropriately for FRET assays.

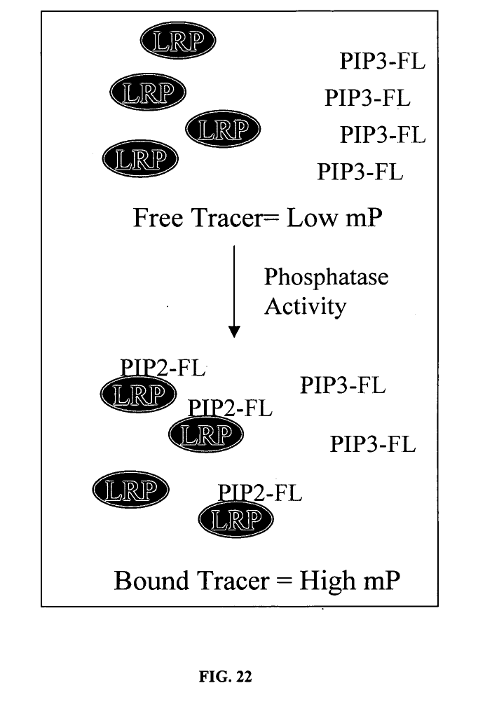

The present invention also provides fluorescence polarization (FP) assays of a lipid phosphatase using fluorescent substrates. Components of this type of assay would include a fluorescently labeled phosphoinositide or inositol phosphate which is able to act as a suitable substrate for the enzyme to be tested, and an antibody or Lipid Recognition Protein with appropriate specificity for the products of the reaction, which will also be fluorescently labeled. In the absence of enzyme activity, the fluorescent molecules will be unbound in solution, resulting in low polarization values. Modification by active enzyme will result in binding of the antibody or detector protein to the fluorescent products of the reaction, resulting in increased polarization values. This is diagrammed in FIG. 22, which illustrates how the fluorescent products of a phosphatase reaction will interact with the detector to produce an increase in polarization values (mP) that is proportional to enzyme activity. The same approach can be used to detect modification of fluorescent phosphoinositide and inositol phosphate substrates by both kinases and phosphatases.

Fluorescence resonance energy transfer (FRET) is a distance-dependent interaction between the electronically excited states of two dye molecules. The donor emits excited-state energy at a wavelength within the excitation spectrum of the acceptor, this energy transfer results in the emission of fluorescence from the acceptor. The emission spectra of the donor and the excitation spectra of the acceptor must have sufficient overlap, as diagrammed on the right, for this to be efficient. The distance between the donor and acceptor molecules is also important, as energy transfer is most efficient over distances of less than 100 Å. FRET has been applied to investigating a variety of biological phenomena that produce changes in molecular proximity.

Time-resolved fluorescence energy transfer (TR-FRET) is a variation of FRET that makes use of long-lived fluorescent molecules as donors. Chelates of rare earth elements (lanthanide chelates) are used as donors, with conventional fluorescent molecules as acceptors. The advantage of using the lanthanide chelates as donors is that their excited-state lifetimes are on the millisecond time scale, while that of most small-molecule fluorophores is on the nanosecond time scale. Thus, a delay of 100 μs before measuring the fluorescence of either the donor or the acceptor species can be used to “gate out” interfering fluorescence arising from matrix components, library compounds or laboratory plastics. This gated detection method also reduces fluorescence signals arising from direct excitation of the acceptor fluorophore. The signal-to-background ratio for TR-FRET is typically several fold higher than is generally seen with standard, shorter lifetime FRET pairs. Additionally, the large Stokes shift of the lanthanide chelates (>200 nm) helps to decrease background fluorescence levels. Table 3 shows some of the donor and acceptor pairs that can be used for FRET and TR-FRET. Most of these are commercially available in formulations suitable for conjugation to proteins or synthetic lipids. Due to its advantages over conventional FRET, the present invention provides reagents and protocols for use in TR-FRET assays for kinase and phosphatase activity.

TABLE 3
Possible Donor and Acceptor Pairs for use in FRET
and TR-FRET assays

| | |
|---|---|
| Donors | Acceptors |

FRET

| | |
|---|---|
| Fluorescein | Tetramethylrhodamine |
| Tetramethylrhodamine | Texas Red |

TR-FRET

| | |
|---|---|
| Europium (Eu) | Cy5 ® |
| | Allophycocyanin (APC) |
| | AlexaFluor ® 647 |
| Terbium (Tb) | Fluorescein |
| | Rhodamine |
| | BODIPY ®-TMR |
| | BODIPY ®-FL |

TR-FRET assays can be implemented as either competitive or as direct assays. In both cases, the assay detects interaction of a lanthanide-derivative labeled antibody or LRP with an acceptor fluorophore-labeled phosphoinositide. In a competitive TR-FRET, the PH domain of an LRP, which specifically binds to the product lipid of a lipid phosphatase, i.e. P(4,5)P₂, can be directly labeled with an Eu chelate. An appropriate phosphoinositide conjugated to CyS® or AlexaFluor® 647 can be used as the binding partner.

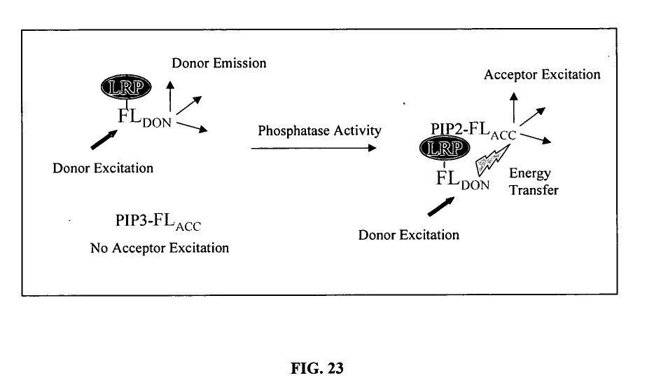

Although the present invention can be implemented as a competitive assay, TR-FRET assays are preferred to detect conversion of a fluorophore-labeled phosphoinositide or inositol phosphate substrate. Components of such an assay are an appropriate phosphoinositide or inositol phosphate substrate labeled with a acceptor fluorophore, and an antibody or Lipid Recognition Protein, labeled with a suitable donor fluorophore, with appropriate specificity for the products of the reaction. In the absence of enzyme activity, there is no interaction of the fluorescent phosphoinositide with the detector protein or antibody, and thus there is no emission by the acceptor fluorophore upon exitation of the donor fluorophore. Upon modification by enzymes, including kinases and phosphatases, the interaction of the LRPs with the reaction products brings the donor and acceptor fluorophores in close enough proximity to generate a FRET signal. FIG. 23 shows a schematic of such an assay applied for the detection of PI(4,5)P₂ produced by dephosphorylation of PI(3,4,5)P₃.

LRPs will be labeled with an Eu chelate, as the donor molecule, according to the manufacturer's protocols (Amersham, or PerkinElmer Life Sciences). AlexaFluor®647-labeled phosphoinositides or inositol phosphates can be used as the acceptor. Alternatively, Biotinylated phosphoinositides complexed to APC-streptavidin are also suitable. Binding curves can be performed to examine the specificity of the interactions of the labeled LRPs, for example, to verify that Eu-TAPP1 interacts specifically with AlexaFluor®647-PI(3,4)P₂ and not AlexaFluor®647-PI(3,4,5)P₃. Because labeling the LRP with Eu could produce conformational changes, it may be necessary to adjust labeling conditions to avoid compromising the specificity of the LRP-phosphoinositide interaction. Once this specificity of the protein-tracer interaction has been demonstrated, the conditions best suited for detection of enzyme activity can be determined in experiments that model substrate conversion under different conditions. In addition, since the activity of lipid phosphatases toward AlexaFluor® and Cy5® labeled phosphoinositides and inositol phosphates has not been demonstrated, the activity of PTEN and SHIP2 enzymes toward these substrates can also be tested.

In addition to the above-described advantages of the assay methods of the present invention, a non-radioactive assay using an LRP as a lipid detection reagent for assaying enzyme activity readily lends itself to automation. Assay platforms used in the non-radioactive assay can be used, for example, with an automatic analyzer such as the Fusion Universal Microplate Analyzer by PerkinElmer Life Sciences. Such a device is easily integrated with automated systems for plate stacking, liquid handling and cell-based assays. Other automation can be applied to the process in order to increase the assay process rate, including a plate washer, harvester and plate scintillation counter, such as Orca/Biomek®.

The principles of the present invention may be applied in a clinical assay setting. A clinical assay can be performed for phosphatase activity that is suitable for analysis of small, less invasive clinical samples, such as blood, pap smears, and needle biopsies. The assay can be applied to samples of cells or biological fluid for direct detection of lipids without performing a lipid extraction. A kit designed for use in a clinical setting can use either an ELISA or fluorogenic format, and would be similar to that designed for use in a research lab.

Another embodiment of the present invention relates to the use of a fluorescence quenching system to detect phospholipids. In essence, this fluorescence quenching system is one form of the FRET assay. The assay system comprises a fluorescently labeled phosphoinositide probe, a fluorescence quencher-tagged lipid recognition protein (LRP) and the phosphoinositide analytes. The phosphoinositide analytes can be the products from the reaction by PI3-K, PTEN and SHIP. In this system, the donor fluorescence donor is the fluorescently labeled phosphoinositide probe that emits excited-state energy at a wavelength within the absorption spectrum of the acceptor, which is the quencher. This energy transfer results in the quenching effects of the fluorescence. The emission spectra of the fluorescent phosphoinositide probe and the absorption spectra of the quencher must have sufficient overlap, as diagrammed in FIG. 23, for this to be efficient. The distance between the donor and quencher molecules is also important, as energy transfer is most efficient over distances of less than 100 Å. An important component of this system is the lipid recognition protein attached with fluorescence quenchers. The incorporation of quenchers may be achieved through two alternative strategies.

1. Small Molecule Quencher-modified LRP

The chemical modification of LRPs, e.g. Grp1, a PI(3,4,5)P₃ binder, with small molecular quenchers (SMQ) has been thoroughly studied in this invention. Three SMQs, Dabcyl-SE (4-((4-(dimethylamino)phenyl)azo)benzoic acid, succinimidyl ester), QSY 7-SE (9-[2-[[4-[[(2,5-dioxo-1-pyrrolidinyl)oxy]carbonyl]-1-piperidinyl]sulfonyl]phenyl]-3,6-bis(methylphenylamino)-, chloride) and QSY 9-SE (QSY® 9 carboxylic acid, succinimidyl ester, C₄₃H₃₉ClN₄O₁₃S₃), with diverse structures, spectral properties, hydrophobicity and solubility properties, were coupled to Grp1. Specifically, SMQ-modified Grp1 still retains its binding activity and quenches the fluorescence of BODIPY®-FL labeled PI(3,4,5)P₃. With a fixed amount of BODIPY®-FL-PI(3,4,5)P₃ in solution, increasing the amount of SMQ-Grp1 caused decreasing fluorescence of the PI(3,4,5)P₃ fluorescent probes. At a ratio of 2:1 (QSY 7-Grp1:BODIPY®-FL-PI(3,4,5)P₃), QSY 7-Grp1 quenched up to 70% of the fluorescence intensity of BODIPY®-FL PI(3,4,5)P₃.

Grp1 conjugation was achieved through the following procedure. 400 μg of Grp1 was conjugated in a 1:10 ratio (SMQ:Grp1) to 40 μg of SMQ using a 0.1 M Sodium Bicarbonate buffer. The concentration of Grp1 in the reaction needed to be >2 mg/ml. The reaction was then rotated for 0.5 hr at 25° C. One purification method for SMQ-Grp1 conjugate was to use GST affinity resins. The initial reaction mixture was bound to 100 μl GST resin pre-equilibrated in PBS (phosphate-buffered saline, pH 7.4) for 1 hr. The bound mix was transferred to a disposable column, washed with 1 ml PBS and eluted in 5×100 μl fractions using PBS and 10 mM glutathione (GSH) at pH 8.0. Fractions were run on SDS-PAGE gel and the target bands were pooled. An alternative purification method for SMQ-Grp1 conjugates was to use dialysis to remove the unreacted quencher molecules. The reaction mixture was dialyzed against PBS for overnight.

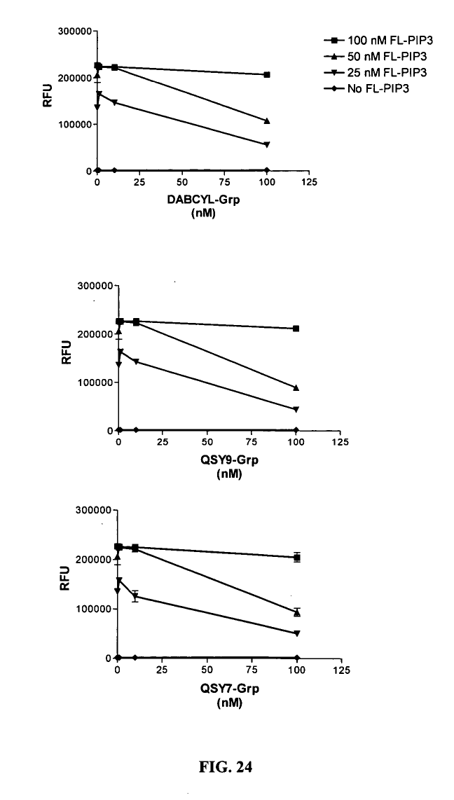

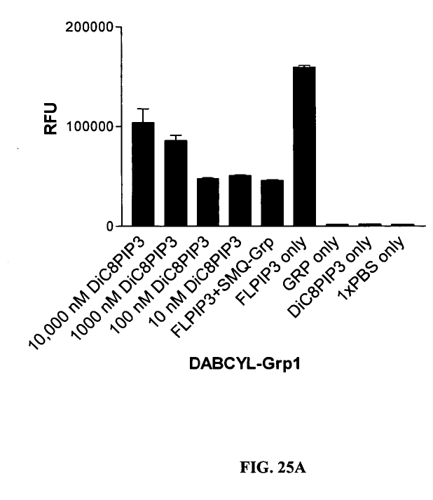

The fluorogenic assays were set up in a black 384-well plate. The combination of 100 nM, 10 nM and 1 nM of SMQ-Grp1 conjugate was incubated with 100 nM, 50 nM and 25 nM BODIPY®-FL-labeled PI(3,4,5)P₃. The total assay volume was 40 PI based in a PBS buffer at a pH of 7.5. The assay mixture was incubated in the dark for one hour at 25° C. The plate was then transferred to an Alpha Fusion plate reader and read with a 485/520 nm filter. As shown in FIG. 24, the SMQ-Grp1 conjugate (Dabcyl-Grp1, QSY 7-Grp1, QSY 9-Grp1) demonstrated fluorescence quenching effects for BODIPY®-FL-PI(3,4,5)P₃. For example, if BODIPY®-FL-PI(3,4,5)P₃ is fixed at 25 nM, increasing the amount of Dabcyl-Grp1 conjugate (from 1 nM to 100 nM) caused decreasing amounts of fluorescence (FIG. 24). Similar fluorescence quenching profiles were shown for QSY 7-Grp1 and QSY 9-Grp1.

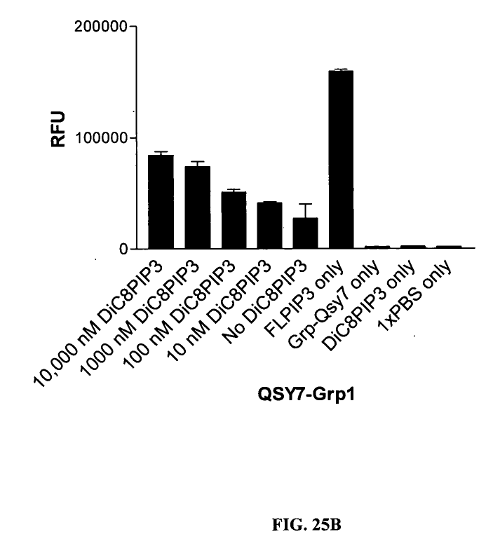

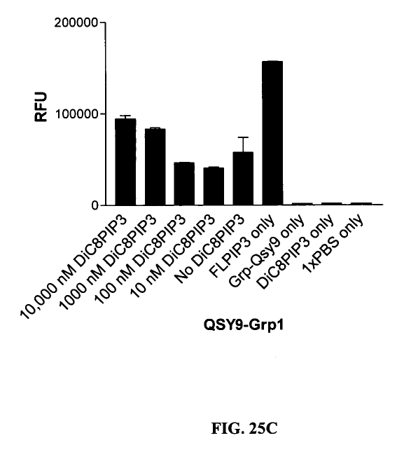

The quenching effects of BODIPY®-FL-PI(3,4,5)P₃ by SMQ-Grp1 were specific. diC₈ PI(3,4,5)P₃ as a competitor antagonized the fluorescence quenching effect of BODIPY®-FL-PI(3,4,5)P₃ by SMQ-Grp1 in a dose-response manner. diC8 PI(3,4,5)P₃ was used at varying concentrations to compete with BODIPY®-FL-PI(3,4,5)P₃ for binding to the SMQ-Grp1 conjugate. The assay system of FIG. 25 was composed of BODIPY®-FL-PI(3,4,5)P₃, SMQ-Grp1, and diC₈ PI(3,4,5)P₃ as the competitor. diC₈ PI(3,4,5)P₃ competed for the binding of BODIPY®-FL-PI(3,4,5)P₃ to SMQ-Grp1 and restored the fluorescence of BODIPY®-FL-PI(3,4,5)P₃ in a dose dependent manner. In each bar graph in FIG. 25, from the right to the left, the bars show the fluorescence reading of PBS as the background, the diC₈ PI(3,4,5)P₃ only, the SMQ-Grp1 conjugate only, BODIPY®-FL-PI(3,4,5)P₃ only, Grp1-SMQ plus BODIPY®-FL-PI(3,4,5)P₃, Grp1-SMQ plus BODIPY®-FL-PI(3,4,5)P₃ with 10 pmol of diC₈ PI(3,4,5)P₃ as the competitor, Grp1-SMQ plus BODIPY®-FL-PI(3,4,5)P₃ with 100 pmol of diC₈ PI(3,4,5)P₃ as the competitor, Grp1-SMQ plus BODIPY®-FL-PI(3,4,5)P₃ with 1000 pmol of diC₈ PI(3,4,5)P₃ as the competitor, and Grp1-SMQ plus BODIPY®-FL-PI(3,4,5)P₃ with 10000 pmol of diC₈ PI(3,4,5)P₃ as competitor. The amount of SMQ-Grp1 present was 100 nM and BODIPY®-FL-PI(3,4,5)P₃ was 50 nM, in each well. Similar competition patterns were obtained for the Grp1-DABCYL, Grp1-QSY7 and Grp1-QSY9 conjugates. As shown in FIG. 25, increasing amounts of diC₈ PI(3,4,5)P₃ caused competition with the BODIPY®-FL-PI(3,4,5)P₃, resulting in an increase in signal. For example, Dabcyl-Grp1 conjugate quenched nearly 70% of the fluorescence of BODIPY®-FL-PI(3,4,5)P₃, but 10000 pmol of diC₈ PI(3,4,5)P₃ restored the fluorescence up to 70% of the original (FIG. 25).

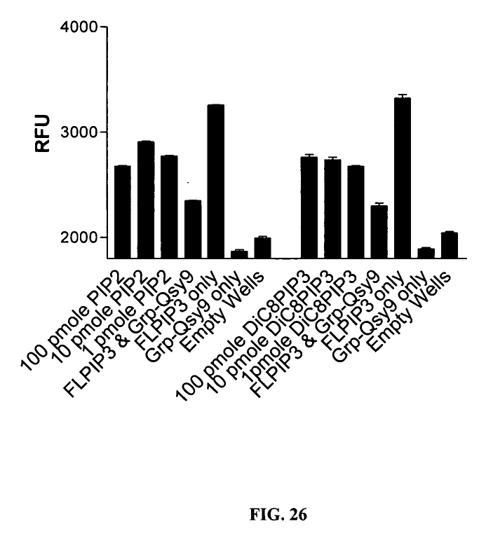

Fluorogenic assays were also used to compare competition of diC₈ PI(3,4,5)P₃ to PI(4,5)P₂ and PI(3)P. With increasing amounts of PI(3,4,5)P₃ the signal begins to increase, indicating competition, whereas as PI(4,5)P₂ and PI(3)P show little change, indicating less competition. The Grp1-SMQ fluorogenic system of the present invention was used to detect PI(3,4,5)P₃ generated in a PI 3-Kinase reaction. 100, 10 and 1 pmol of PI(4,5)P₂ were incubated with PI 3-Kinase at 25° C. for 3 hours. The products were then incubated with 1000 pmol BODIPY®-FL-PI(3,4,5)P₃ and 50 nM Grp1-QSY 9. The bars in FIG. 26, from the right to left, show the fluorescence reading of empty wells, Grp1-QSY9 only, BODIPY®-FL-PI(3,4,5)P₃ only, Grp1-QSY9 plus BODIPY®-FL-PI(3,4,5)P₃, Grp1-QSY9 plus BODIPY®-FL-PI(3,4,5)P₃ with 1 pmol of diC₈ PI(3,4,5)P₃ as the competitor, Grp1-QSY9 plus BODIPY®-FL-PI(3,4,5)P₃ with 10 pmol of diC₈ PI(3,4,5)P₃ as the competitor and Grp1-QSY9 plus BODIPY®-FL-PI(3,4,5)P₃ with 100 pmol of diC₈ PI(3,4,5)P₃ as the competitor; empty wells, Grp1-QSY9 only, BODIPY®-FL-PI(3,4,5)P₃ only, Grp1-QSY9 plus BODIPY®-FL-PI(3,4,5)P₃, Grp1-QSY9 plus BODIPY®-FL-PI(3,4,5)P₃ with diC8 PI(3,4,5)P₃ produced by PI 3-Kinase from 1 pmol of diC₈ PI(4,5)P₂ as the competitor, Grp1-QSY9 plus BODIPY®-FL-PI(3,4,5)P₃ with diC₈ PI(3,4,5)P₃ produced by PI 3-Kinase from 10 pmol of diC₈ PI(4,5)P₂ as the competitor and Grp1-QSY9 plus BODIPY®-FL-PI(3,4,5)P₃ with diC₈ PI(3,4,5)P₃ produced by PI 3-Kinase from 100 pmol of diC₈ PI(4,5)P₂ as the competitor. The results in FIG. 26 show that PI(3,4,5)P₃ produced by PI 3-Kinase from PI(4,5)P₂ has similar competitive ability in this fluoregenic system. This means that the fluorogenic assay can be used as a detection system to monitor the production of PI(3,4,5)P₃ by PI 3-Kinase from PI(4,5)P₂. The production (or presence) of PI(3,4,5)P₃ in this assay system corresponds to the elevation of the fluorescence signal.

2. Small Molecular Quencher-modified Glutathione

A dye-tethered glutathione (GSH) derivative can serve as a universal direct detector of GST-tagged fusion proteins. This is based on the hypothesis that such GS-dye analogs can still bind to GST. Specifically, the free thiol of the glutathione molecule was reacted to a thiol-reactive chromophore (e.g. QSY 9, maleimide). The maleimide group reacted to the free thiol group of glutathione to produce a glutathione-chromophore conjugate. The glutathione-chromophore conjugate was demonstrated to still retain its binding ability to GST-tagged proteins. The general preparation procedure of chromophore-labeled GSH is exemplified as follows: GSH (290 μg, 130 μM) was dissolved in 600 μl of PBS which was previously degassed for one hour. BODIPY®-FL-Maleimide (32 μl, 10×excess of GSH) was added to the solution dropwise. After stirring for 2 hours, the reaction mixture was extracted six times by ethyl acetate (EtOAc) of 500 μl. The aqueous phase was subjected to SpeedVace to remove any traces of EtOAc, and β-mercaptoethanol (10 μM) was added for 30 minutes to quench the free maleimide.

Two glutathione conjugates, glutathione-BODIPY®-FL and glutathione-QSY 7, have been prepared. The conjugates were purified by reverse phase columns and characterized by MALDI MS. The absorption spectra of these conjugates were obtained and the conjugates ere quantified by UV/Vis absorption according to Beer's Law. One aspect of the present invention relates to the use of a chromophore-labeled GSH to detect GST-tagged proteins either in solution or on a solid support. It is demonstrated in the present invention that the Glutathione-BODIPY®-FL conjugate (GS-BODIPY®) is able to bind to GST-LRP blotted on a nitrocellulose membrane. In a separate experiment, Grp1 that was bound to PI(3,4,5)P₃ plates was detected by glutathione-BODIPY®-FL with considerable sensitivity and specificity.

The fluorogenic assays using a glutathione quencher conjugate (e.g. GS-QSY 7) were set up in a black 384-well plate. A combination of the assay components described in detail below, including GST-Grp1, BODIPY®-FL-PI(3,4,5)P₃, GS-QSY7, and phosphoinositide competitors, were incubated in the dark for 1 hour. The total reaction volume was either 25 or 50 μl in PBS (pH 7.4). The plate was then transferred to an Alpha Fusion plate reader and read with a 485/520 nm filter.

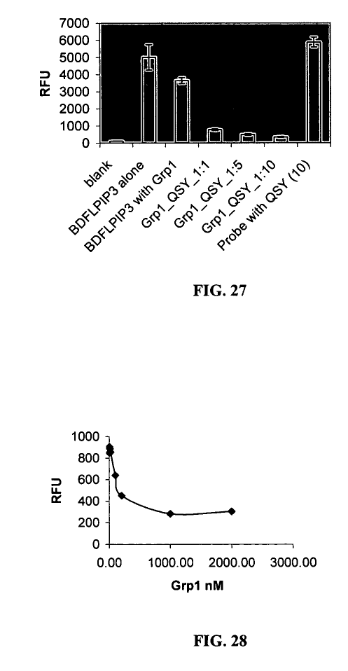

As illustrated in FIG. 27, with BODIPY®-FL-PI(3,4,5)P₃ (10 nM) and GST-Grp1 (0.6 μM), GS-QSY7 at increasing concentrations showed increasing quenching effects with GST-Grp1:GS-QSY7 ratios of 1:1, 1:5 and 1:10. In the bar graph of FIG. 27, from left to right, the bars shows the fluorescence reading of PBS as blank, BODIPY®-FL-PI(3,4,5)P₃ alone, BODIPY®-FL-PI(3,4,5)P₃ plus GST-Grp1, BODIPY®-FL-PI(3,4,5)P₃ plus GST-Grp1 and GS-QSY 7 (GST-Grp1:GS-QSY 7=1:1), BODIPY®-FL-PI(3,4,5)P₃ plus GST-Grp1 and GS-QSY 7 (GST-Grp1:GS-QSY 7=1:5), BODIPY®-FL-PI(3,4,5)P₃ plus GST-Grp1 and GS-QSY 7 (GST-Grp1:GS-QSY 7=1:10), and BODIPY®-FL-PI(3,4,5)P₃ plus GS-QSY 7 (BODIPY®-FL-PI(3,4,5)P₃:GS-QSY 7=1:600). Fluorescence probes with Grp1 or GS-QSY7 alone did not show significant fluorescence quenching effects. This indicates that the fluorescence of BODIPY®-FL-PI(3,4,5)P₃ was quenched efficiently by the GS-QSY 7/GST-Grp1 complex.

As illustrated in FIG. 28, with a fixed amount of BODIPY®-FL-PI(3,4,5)P₃ (10 nM) and GS-QSY 7 (1000 nM), increasing the amount of GST-Grp 1 offers better fluorescence quenching effects with a ratio (BODIPY®-FL-PI(3,4,5)P₃ :GST:Grp1) at 1:50 reaching the lower plateau of fluorescence. This indicates that the fluorescence of BODIPY®-FL-PI(3,4,5)P₃ was quenched by the GST-Grp1/GS-QSY 7 complex in a dose dependent manner.

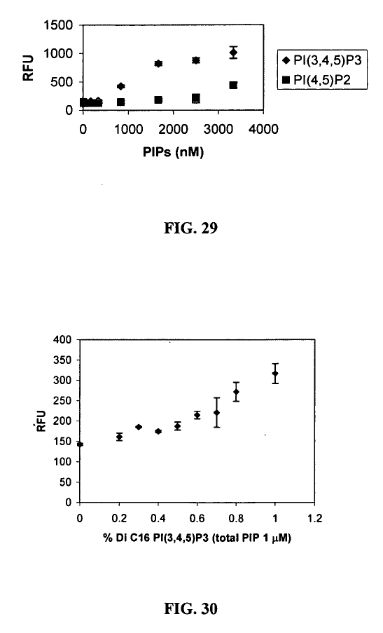

This fluorogenic assay was also specific. FIG. 29, shows the fluorescence readings of BODIPY®-FL PI(3,4,5)P₃ at 10 nM, GST-Grp1 at 0.5 μM, GS-QSY 7 at 2.5 μM with an increasing amount of diC₁₆ PI(3,4,5)P₃ as a competitor. DiC₁₆ PI(3,4,5)P₃ restored the fluorescence of BODIPY®-FL PI(3,4,5)P₃, while diC₁₆ PI(4,5)P₂ only marginally increased the fluorescence at higher concentrations. This indicates that diC₁₆ PI(3,4,5)P₃ specifically restored the fluorescence while diC₁₆ PI(4,5)P₂ only demonstrated marginal competition at higher concentrations. The clear differentiation of diC₁₆ PI(3,4,5)P₃ and diC₁₆ PI(4,5)P₂ indicates that this assay system is fully capable of detecting the production of diC₁₆ PI(3,4,5)P₃ by PI 3-Kinase and the production of diC₁₆ PI(4,5)P₂ by PTEN.

This notion is further illustrated in FIG. 30, in a mixture of diC₁₆ PI(4,5)P₂ and PI(3,4,5)P₃ that mimics the conversion of PI(4,5)P₂ to PI(3,4,5)P₃ in a PI 3-Kinase reaction, the fluorescence was restored with an increasing percentage of PI(3,4,5)P₃. Specifically, the total phosphoinositides, PI(4,5)P₂ and PI(3,4,5)P₃, in each well is 1 μM, which means if PI(3,4,5)P₃ is 20% at 0.2 μM, PI(4,5)P₂ is 80% at 0.8 μM. Fluorescence of BODIPY®-FL-PI(3,4,5)P₃ was gradually restored with increasing amounts of PI(3,4,5)P₃. The other components in this assay were BODIPY®-FL PI(3,4,5)P₃ at 10 nM, GST-Grp1 at 0.5 μM and GS-QSY7 at 2.5 μM.

Therefore, the quencher-LRP conjugates of the present invention specifically suppressed the fluorescence of the phosphoinositide probe and the presence of lipid analytes competed for binding and restored the fluorescence in a dose-dependent manner. The fluorogenic assay of the present invention can be easily upgraded to a high throughput format for lipid-metabolizing enzymes or can be adapted to analyze other metabolites of interest with the following advantages. First, the assay is homogenous and is a mix-and-measure method, which means the assay does not require washing steps, only mix the components and measure the outcome. Second, the presence of analytes causes a positive signal. Third, the assay is robust and has high sensitivity. Finally, glutathione conjugation offers a new approach for modification of GST-tagged proteins. Modification of a protein does not need to occur randomly at the reactive amino acid residues, which may comprise the biological activities of the Lysine or Cystine residues, but can be engineered in a more site-specific way.

The preceding description has been presented only to illustrate and describe the invention. The preferred embodiment was chosen and described in order to best explain the principles of the invention and its practical application. The preceding description is intended to enable others skilled in the art to best utilize the invention in various embodiments and with various modifications as suited to the particular use contemplated. It will be apparent to those of ordinary skill in the art that numerous modifications can be made without departing from the principles and concepts of the invention as set forth in the claims.

1. A lipid assay method comprising the steps of: exposing a lipid detector protein or antibody with a binding specificity for a product lipid of a lipid kinase or phosphatase to a solution containing said product lipid or a reaction mixture of a substrate lipid and said lipid kinase or phosphatase; and quantifying said product lipid in said solution.

2. The lipid assay method according to claim 1, wherein said lipid detector protein is a lipid recognition protein (LRP) which contains an affinity tag fusion with a pleckstrin homology (PH) domain or other lipid-binding domains.

3. The lipid assay method according to claim 1, wherein said assay is a plate-based assay.

4. The lipid assay method according to claim 3, wherein said assay plate is coated with streptavidin, glutathione or Protein A.

5. The lipid assay method according to claim 1, wherein said assay is an enzyme linked immunosorbent assay (ELISA) or an amplified luminescence proximity homogenous assay (ALPHA).

6. The lipid assay method according to claim 1, wherein said assay is a fluorogenic assay selected from the group consisting of fluorescence polarization (FP) assay, fluorescence resonance energy transfer (FRET) assay and time-resolved fluorescence resonance energy transfer (TR-FRET) assay.

7. The lipid assay method according to claim 1, wherein additional lipids or inositol phosphates are present in said solution.

8. The lipid assay method according to claim 1, wherein said substrate lipid is a member selected from the group consisting of PI(3,4,5)P₃, PI(3,4)P₂, PI(3,5)P₂, PI(4,5)P₂, PI(3)P, PI(4)P, PI(5)P, I(1,3,4,5)P₄, I(1,3,4)P₃, I(1,3,5)P₃, I(1,4,5)P₃, I(1,3)P₂, I(1,4)P₂ and I(1,5)P₂.

9. The lipid assay method according to claim 1, wherein said product lipid is a member selected from the group consisting of PI(3,4)P₂, PI(4,5)P₂, PI(3,5)P₂, PI(3)P, PI(4)P, PI(5)P, I(1,3,4,5)P₄, I(1,3,4)P₃, I(1,3,5)P₃, I(1,4,5)P₃, I(1,3)P₂, I(1,4)P₂ and I(1,5)P₂.

10. The lipid assay method according to claim 1, wherein the assay is a direct assay wherein said product lipid has a stronger affinity to said lipid detector protein or antibody than said substrate lipid.

11. The lipid assay method according to claim 10, wherein said substrate lipid is modified via conjugation of a fluorophore and the fluorescent signal produced is directly proportional to the amount of product generated.

12. The lipid assay method according to claim 10, wherein said substrate lipid is modified by biotinylation.

13. The lipid assay method according to claim 1, wherein said substrate lipid is immobilized onto the surface of a multi-well assay plate or bead.

14. The lipid assay method according to claim 1, wherein the assay is a competitive assay wherein said lipid detector protein or antibody is further exposed to a probe lipid and a competition occurs between said product lipid and said probe lipid.

15. The lipid assay method according to claim 14, wherein said probe lipid is modified via conjugation of fluorophores and the fluorescent signal produced is inversely proportional to the amount of said lipid product.

16. The lipid assay method according to claim 14, wherein said probe lipid is modified by biotinylation.

17. The lipid assay method according to claim 14, wherein said wherein said probe lipid is immobilized onto the surface of a multi-well assay plate or a bead.

18. The lipid assay method according to claim 14, wherein said probe lipid is a member selected from the group consisting of PI(3,4)P₂, PI(4,5)P₂, PI(3,5)P₂, PI(3)P, PI(4)P, PI(5)P, I(1,3,4,5)P₄, I(1,3,4)P₃, I(1,3,5)P₃, I(1,4,5)P₃, I(1,3)P₂, I(1,4)P₂ and I(1,5)P₂.

19. The lipid assay method according to claim 2, wherein said lipid recognition protein (LRP) is tagged with a small molecular quencher (SMQ) or a small molecular quencher (SMQ) modified glutathione.

20. The lipid assay kit according to claim 19, wherein said small molecular quencher (SMQ) is Dabcyl-SE, QSY 7-SE or QSY 9-SE.

21. A lipid assay kit comprising: a lipid detector protein or antibody containing a lipid recognition motif with a binding specificity for a product lipid of a lipid kinase or phosphatase, and a solution containing a substrate lipid or inositol phosphate of said lipid kinase or phosphatase.

22. The lipid assay kit according to claim 21 is a direct assay kit wherein said product lipid has a stronger affinity to said lipid detector protein or antibody than said substrate lipid.

23. The lipid assay kit according to claim 22, wherein said substrate lipid is modified via conjugation of a fluorophore and the fluorescent signal produced is directly proportional to the amount of lipid product.

24. The lipid assay kit according to claim 22, wherein said substrate lipid is modified by biotinylation.

25. The lipid assay kit according to claim 22, further comprising a multi-well assay plate or a bead and wherein said substrate lipid is non-radioactively labeled and is immobilized to the wall of said plate or said bead.

26. The lipid assay kit according to claim 21 is a competitive assay kit wherein said lipid detector protein or antibody is further exposed to a probe lipid and a competition occurs between said product lipid and said probe lipid.

27. The lipid assay kit according to claim 26, wherein said probe lipid is modified via conjugation of a fluorophore and the fluorescent signal produced is inversely proportional to the amount of lipid product.

28. The lipid assay kit according to claim 26, wherein said probe lipid is modified by biotinylation.

29. The lipid assay kit according to claim 26, further comprising a multi-well assay plate or a bead and wherein said probe lipid is non-radioactively labeled and is immobilized to the well of said plate or said bead.

30. The lipid assay kit according to claim 29, wherein said assay plate or bead is coated with streptavidin, glutathione or Protein A.

31. The lipid assay kit according to claim 21, wherein said lipid detector protein is a lipid recognition protein (LRP) which contains an affinity tag fusion with a pleckstrin homology (PH) domain or other lipid-binding domains.

32. The lipid assay kit according to claim 21, wherein said lipid recognition protein is an antibody which interacts specifically with said product lipid.

33. The lipid assay kit according to claim 21, wherein said assay is an enzyme linked immunosorbent assay (ELISA) or an amplified luminescence proximity homogenous assay (ALPHA).

34. The lipid assay kit according to claim 21, wherein said assay is a fluorogenic assay selected from the group consisting of a fluorescence polarization (FP) assay, a fluorescence resonance energy transfer(FRET) assay and a time-resolved fluorescence resonance energy transfer(TR-FRET) assay.

35. The lipid assay kit according to claim 21, wherein additional lipids or inositol phosphates are present in said solution.

36. The lipid assay kit according to claim 21, wherein said substrate lipid is a member selected from the group consisting of PI(3,4,5)P₃, PI(3,4)P₂, PI(3,5)P₂, PI(4,5)P₂, PI(3)P, PI(4)P, PI(5)P, I(1,3,4,5)P₄, I(1,3,4)P₃, I(1,3,5)P₃, I(1,4,5)P₃, I(1,3)P₂, I(1,4)P₂ and I(1,5)P₂.

37. The lipid assay kit according to claim 21, wherein said product lipid is a member selected from the group consisting of PI(3,4)P₂, PI(4,5)P₂, PI(3,5)P₂, PI(3)P, PI(4)P, PI(5)P, I(1,3,4,5)P₄, I(1,3,4)P₃, I(1,3,5)P₃, I(1,4,5)P₃, I(1,3)P₂, I(1,4)P₂ and I(1,5)P₂.

38. The lipid assay kit according to claim 26, wherein said probe lipid is a member selected from the group consisting of PI(3,4)P₂, PI(4,5)P₂, PI(3,5)P₂, PI(3)P, PI(4)P, PI(5)P, I(1,3,4,5)P₄, I(1,3,4)P₃, I(1,3,5)P₃, I(1,4,5)P₃, I(1,3)P₂, I(1,4)P₂ and I(1,5)P₂.

39. The lipid assay kit according to claim 31, wherein said lipid recognition protein (LRP) is tagged with a small molecular quencher (SMQ) or a small molecular quencher (SMQ) modified glutathione.

40. The lipid assay method according to claim 39, wherein said small molecular quencher (SMQ) is Dabcyl-SE, QSY 7-SE or QSY 9-SE.

41. An assay kit for detecting a lipid-metabolizing enzyme activity comprising: a lipid metabolizing enzyme, a substrate phosphoinositide of said enzyme, a product phosphoinositide of said enzyme, a fluorescently labeled phosphoinositide probe and a fluorescence quencher-tagged lipid recognition protein (LRP) with a binding specificity for said product phosphoinositide.

42. The lipid assay kit according to claim 41, wherein said lipid recognition protein (LRP) is tagged with a small molecular quencher (SMQ) or a small molecular quencher (SMQ) modified glutathione.

43. The lipid assay kit according to claim 42, wherein said small molecular quencher (SMQ) is Dabcyl-SE, QSY 7-SE or QSY 9-SE.

44. The assay kit according to claim 41, wherein said lipid-metabolizing enzyme is a phosphoinositide kinase or a phosphoinositide phosphatase.

45. The assay kit according to claim 44, wherein said product phosphoinositide is a member selected from the group consisting of PI(3,4,5)P₃, PI(3,4)P₂, PI(3,5)P₂, PI(4,5)P₂, PI(3)P, PI(4)P, PI(5)P and PI.
